(19) United States
(12) Patent Application Publication
(10) Pub. No.: US 2025/0108068 A1
(43) Pub. Date: Apr. 3, 2025

(54) COMPOSITIONS AND METHODS FOR ALLEVIATING PULMONARY AILMENTS

(71) Applicant: Color Ventures LLC, Washington, DC (US)

(72) Inventors: Nick Haft, Washington, DC (US); Zachary Warnken, Austin, TX (US); Dale J. Christensen, Moneta, VA (US)

(73) Assignee: Color Ventures LLC, Washington, DC (US)

(21) Appl. No.: 18/900,293

(22) Filed: Sep. 27, 2024

Related U.S. Application Data
(60) Provisional application No. 63/541,247, filed on Sep. 28, 2023.

(51) Int. Cl. A61K 33/00; A61K 9/00; A61K 31/194; A61K 45/06

(52) U.S. Cl. CPC A61K 33/00; A61K 9/0075; A61K 31/194; A61K 45/06

(57) ABSTRACT

This disclosure relates to compositions suitable for administration to a subject by inhalation comprising cysteine, citric acid or a physiologically acceptable salt thereof, carbonic acid or a physiologically acceptable salt thereof, and leucine, and methods of use. The disclosed compositions are useful for alleviating one or more negative effects of a pulmonary or airway disorder. In addition, the disclosed compositions are useful for enhancing clearance of pollutants, microparticulate matter, or other irritants from the lungs of a subject. Finally, the disclosed compositions are useful for cleansing at least a portion of the lungs of a subject.

### CROSS-REFERENCE TO RELATED PATENT APPLICATION

This application claims the benefit under 35 U.S.C. § 119(e) of U.S. Provisional Application No. 63/541,247, filed Sep. 28, 2023, the entire contents of which are incorporated herein by reference.

### BACKGROUND

A large unmet medical need exists for safe, well-tolerated and effective compositions for the treatment of patients with pulmonary ailments characterized by overproduction of thickened mucus resulting in impaired lung function such as irritant/pollutant-induced lung disorders, cystic fibrosis (CF), COPD/emphysema, bronchiectasis, severe asthma and other obstructive pulmonary diseases that are generally localized in a patient's lungs. Poor clearance of abnormal, sticky mucus is associated with chronic infection and premature death. Despite advances in antibiotic therapy and other treatments, improved mucus clearance remains a central clinical treatment objective. Therefore, there remains a need for compositions that can cleanse lungs (e.g., of mucus, foreign particles and/or toxins) and alleviate pulmonary ailments.

### SUMMARY OF THE INVENTION

One embodiment of the invention relates to a composition comprising: (a) a thiol-containing reducing agent; (b) citric acid or a physiologically acceptable salt thereof; (c) carbonic acid or a physiologically acceptable salt thereof; and (d) a stabilizer.

In some embodiments, the thiol-containing reducing agent comprises cysteine, N-Acetyl cysteine, homocysteine, dihydrolipoic acid, glutathione, coenzyme A, ergothioneine, mycothiol, bacillithiol, or combinations thereof.

In some embodiments, the stabilizer comprises leucine, lecithin, magnesium stearate, trileucine, isoleucine, glycine, lactose, mannitol, trehalose, erythritol, sorbitol, or combinations thereof.

In some embodiments, the molar ratio of (a):(b):(c):(d) ranges from about 0.5-1:0.5-1:0.5-1:0.5-1.

In some embodiments, the composition comprises (a) 40%-80% (w/w) of a thiol-containing reducing agent; (b) 8%-20% (w/w) of citric acid or a physiologically acceptable salt thereof; (c) 8%-20% (w/w) of carbonic acid or a physiologically acceptable salt thereof; and (d) 3%-6% (w/w) of a stabilizer. In some embodiments, the composition comprises a mixture arising from a combination of the aforementioned components, i.e., thiol-containing reducing agent, citric acid, carbonic acid, a stabilizer, or physiologically acceptable salts, if any, of any of the foregoing.

In some embodiments, the composition further comprises a physiologically acceptable carrier and may further comprise one or more additional components, as might be described vide infra.

In some embodiments, the pH of the composition ranges from about 5.5 to about 10, about 5.5 to about 8, about 6 to about 10, or about 6 to about 8.

In some embodiments, the composition comprises a solution of a mixture of components, aqueous or otherwise. In some embodiments the composition comprises a solid mixture, such as a dry powder.

In some embodiments, the composition is suitable for administration by inhalation.

In some embodiments, the composition further comprises at least one ingredient selected from Adhatoda vasica Nees extract, Albizzia lebbeck (Sareesha_rakat) extract, Alstonia scholaris extract, Artemisia caerulescens extract, Belamcanda chinensis extract, Benincasa hispida extract, Cissampelos sympodialis extract, Clerodendron serratum extract, Coleus forskohlii extract, Elaeocarpus spharicus extract, Galphimia glauca extract, Gardenia latifolia extract, Gingko biloba extract, Mikania glomerata extract, Lepidium sativum extract, Ocimum sanctum extract, Passiflora incarnata extract, Pavetta crassipes extract, Picrorrhiza kurroa extract, Sarcostemma brevistigma extract, Tephrosia purpurea extract, Tylophora indica extract, Vitex negundo extract, Rosmarinus officinalis extract, Ephedra sinica extract, and Gleditsia sinensis Lam extract.

In some embodiments, the composition further comprises at least one ingredient selected from bicarbonate, Ascorbic acid, EDTA, N-acetylcysteine, Caffeine citrate, L-Arginine, Forskolin, Theobromine, resolving D1 (RvD1), resolving E1 (RvE1), lipoxin A1 (LXA4), lipoxin B4 (LXB4), protectin D1 (PD1), maresin 1 (MaR1), maresin 2 (MaR2), 17-HDHA, 14-HDHA, ellagic acid, curcumin, 18-HEPE, or a combination thereof.

In some embodiments, the composition comprises a lyophilized dry powder. In some embodiments, the lyophilized dry powder is produced by spray drying or by air jet milling (aka. “top-down approach”).

Another aspect of the disclosure is directed to a dry powder composition suitable for administration to a human subject by inhalation comprising (a) cysteine, (b) citric acid or a physiologically acceptable salt thereof, (c) carbonic acid or a physiologically acceptable salt thereof, and (d) leucine.

In some embodiments, the molar ratio of (a):(b):(c):(d) ranges from about 0.5-1:0.5-1:0.5-1:0.5-1.

In some embodiments, the dry powder composition comprises a mixture arising from a combination of (a) 40%-80% (w/w) of a thiol-containing reducing agent; (b) 8%-20% (w/w) of citric acid or a physiologically acceptable salt thereof; (c) 8%-20% (w/w) of carbonic acid or a physiologically acceptable salt thereof; and (d) 3%-6% (w/w) of a stabilizer.

In some embodiments, the composition is packaged into an inhaler.

In some embodiments, the inhaler is a dry powder inhaler (DPI).

Another aspect of the disclosure is directed to a method of alleviating one or more negative effects of a pulmonary or airway disorder comprising administering to a subject in need thereof a composition arising from a combination of a thiol-containing reducing agent, citric acid or a physiologically acceptable salt thereof, carbonic acid or a physiologically acceptable salt thereof, and a stabilizer.

In some embodiments, the pulmonary or airway disorder is selected from chronic inflammatory lung disease, pulmonary fibrosis, pulmonary vasculitis, pulmonary sarcoidosis, inflammation and/or infection associated with lung transplantation, acute or lung rejection and/or dysfunction, pulmonary artery hypertension, bronchitis, sinusitis, asthma, cystic fibrosis, bronchiectasis, bacterial infection, fungal infection, parasite infection, viral infection, chronic obstructive pulmonary disease (COPD), bronchiolitis obliterans syndrome (BOS), primary ciliary dyskinesia (PCD), alveolar proteinosis, idiopathic pulmonary fibrosis, eosinophilic pneumonia, eosinophilic bronchitis, acute respiratory distress syndrome (ARDS), inflammation and/or infection associated with mechanical ventilation, ventilator-associated pneumonia, asbestos-related airway disorder or disease, dust-related airway disorder or disease, silicosis, and radiation or chemical agent-related airway disease or disorder, and any combination thereof.

Another aspect of the disclosure is directed to a method of enhancing clearance of pollutants, microparticulate matter, or other irritants from the lungs of a subject comprising administering to a subject in need thereof a composition arising from a combination of a thiol-containing reducing agent, citric acid or a physiologically acceptable salt thereof, carbonic acid or a physiologically acceptable salt thereof, and a stabilizer.

Another aspect of the disclosure is directed to a method of cleansing at least a portion of the lungs of a subject comprising administering to at least a portion of the lungs of a subject in need thereof a composition arising from a combination of a thiol-containing reducing agent, citric acid or a physiologically acceptable salt thereof, carbonic acid or a physiologically acceptable salt thereof, and a stabilizer.

In some embodiments, the composition is a dry powder administered through inhalation.

In some embodiments, the composition is administered by a dry powder inhaler (DPI).

In some embodiments, the composition is administered at least once a day, twice a day, three times a day, or as needed.

In some embodiments, the combination excludes divalent cations.

In some embodiments, the composition further comprises a pharmacologically acceptable carrier.

In some embodiments, said at least a portion of the lungs is cleansed of at least one irritant.

In some embodiments, the at least one irritant comprises a cigarette smoke, bacteria, a mold, dust mites, acarids, a pollen, a material from an insect, a material from an animal and animal dander, fungi, an air pollutant, an aerosol, a fume, a chemical, or combinations thereof.

In some embodiments, the composition further comprises at least one ingredient selected from Adhatoda vasica Nees extract, Albizzia lebbeck (Sareesha rakat) extract, Alstonia scholaris extract, Artemisia caerulescens extract, Belamcanda chinensis extract, Benincasa hispida extract, Cissampelos sympodialis extract, Clerodendron serratum extract, Coleus forskohlii extract, Elaeocarpus spharicus extract, Galphimia glauca extract, Gardenia latifolia extract, Gingko biloba extract, Mikania glomerata extract, Lepidium sativum extract, Ocimum sanctum extract, Passiflora incarnata extract, Pavetta crassipes extract, Picrorrhiza kurroa extract, Sarcostemma brevistigma extract, Tephrosia purpurea extract, Tylophora indica extract, Vitex negundo extract, Rosmarinus officinalis extract, Ephedra sinica extract, and Gleditsia sinensis Lam extract.

In some embodiments, the composition further comprises at least one ingredient selected from bicarbonate, Ascorbic acid, EDTA, N-acetylcysteine, Caffeine citrate, L-Arginine, Forskolin, Theobromine, resolving D1 (RvD1), resolving E1 (RvE1), lipoxin A1 (LXA4), lipoxin B4 (LXB4), protectin D1 (PD1), maresin 1 (MaR1), maresin 2 (MaR2), 17-HDHA, 14-HDHA, ellagic acid, curcumin, 18-HEPE, or a combination thereof.

In some embodiments, the thiol-containing reducing agent comprises cysteine, N-Acetyl cysteine, homocysteine, dihydrolipoic acid, glutathione, coenzyme A, ergothioneine, mycothiol, bacillithiol, or combinations thereof.

In some embodiments, the stabilizer comprises leucine, lecithin, magnesium stereate, trileucine, isoleucine, glycine, lactose, mannitol, trehalose, erythritol, sorbitol, or combinations thereof.

### BRIEF DESCRIPTION OF THE DRAWINGS

FIG. 1 shows the timeline and the setup for multiple endpoint analysis. CBF: Cilia Beating Frequency, MMC: Mucociliary Clearance, TEER: Trans-epithelial electrical resistance, LDH: Lactate dehydrogenase, IL-8: interleukin 8.

FIGS. 2A-2D. Scanning electron microscopy results. (A) 1,500× magnification of Formulation 1; (B) 3500× magnification of Formulation 1; (C) 1,500× magnification of Formulation 4; (D) 3500× magnification of Formulation 4.

FIG. 3. Tissue integrity measurements of test epithelia after apical exposure of formulations to test epithelia. Transepithelial electrical resistance (TEER) measurements were obtained to determine the effect of treatments on the integrity of the monolayer of cells and tight junctions.

FIG. 4. Cytotoxicity of formulations on test epithelia after apical exposure to test epithelia. Quantitation of lactate dehydrogenase (LDH) was used to determine the effect of treatments on the monolayer of cells with release of LDH indicative of cell lysis and cytotoxicity.

FIG. 5. Cilia beating frequency of test epithelia after apical exposure of formulations to test epithelia. Video microscopy was used to determine the effect of treatments on the ciliary beat frequency with image processing in each culture monolayer to quantify the movement of cilia.

FIG. 6. Percentage of active area of cilia beating measurements on test epithelia. Video microscopy was used to determine the effect of treatments on the ciliary beat frequency with image processing in each culture monolayer to quantify the percentage of the area in the images that demonstrate ciliary movement.

FIG. 7. Mucociliary clearance of test epithelia after apical exposure of formulations to test epithelia. Video imaging was used to measure the distance that latex microbeads move over time and the velocity in micrometers moved per minute are plotted for a representative sample of beads applied to three separate monolayer airway epithelial cultures.

FIG. 8. Basal secretion of IL-8 by test epithelia after apical exposure of formulations to test epithelia. Media from the basolateral chamber was tested using ELISA to quantify IL-8 as a surrogate for inflammatory signaling caused by the addition of test compounds to the apical side of the monolayer. Results are plotted in ng/mL of the basolateral fluid tested.

FIG. 9A-9C. Formulation 5 L tests. (A) Ciliary beat frequency, (B) Mucociliary clearance, and (C) Toxicity after different treatment with different dilutions of the 5 L formulation and controls.

FIG. 10. Change in the percent of basal Tracheal mucus velocity (TMV) under different treatments.

### DETAILED DESCRIPTION

### Definitions

As it would be understood, the section or subsection headings as used herein is for organizational purposes only and are not to be construed as limiting and/or separating the subject matter described.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, agents, compositions and methods include the recited elements, but not exclude others. “Consisting essentially of” when used to define compounds, agents, compositions, and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

As will be understood by one skilled in the art, for any and all purposes, all ranges disclosed herein also encompass any and all possible subranges and combinations of subranges thereof. Furthermore, as will be understood by one skilled in the art, a range includes each individual member.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

As used herein, the phrase “at least one” refers to one, two, three, four or more (including all) of the listed items.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. A composition is also meant to describe a mixture that results from a combination of two or more components, including any reaction products arising from such a combination (i.e., the term is meant to capture a combination in which the original components may not necessarily be isolatable or detectable in the resulting mixture).

Carriers also include pharmaceutical, dietary supplements, food and cosmetic excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A composition as disclosed herein can be a pharmaceutical composition, for prescription or non-prescription use. A “pharmaceutical composition” is intended to include the combination of an active agent with a carrier, inert or active, making the composition suitable for diagnostic or therapeutic use in vitro, in vivo or ex vivo. A composition as disclosed herein can also be a cosmetic product, which are intended for cosmetic uses. A composition as disclosed herein can also be a dietary supplement or a food, which is intended for consumption.

As used herein, the phrase “pharmaceutically acceptable carriers” or “physiologically acceptable carrier” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, a “physiologically acceptable salt” refers to inorganic or organic salts, including, but not limited to, buffer salts which are not deleterious to cell health or integrity. Physiologically acceptable salts are often useful because they may have improved stability and/or solubility in pharmaceutical compositions over the free base form of the compound. A physiologically acceptable salt may be obtained by reaction of a free base with an inorganic acid such as hydrochloric acid, hydrobromic acid, nitric acid, phosphoric acid, sulfuric acid, and perchloric acid and the like, or with an organic acid such as acetic acid, oxalic acid, malic acid, maleic acid, methanesulfonic acid, ethanesulfonic acid, p-toluenesulfonic acid, salicylic acid, tartaric acid, citric acid, succinic acid or malonic acid, and the like. A physiologically acceptable salt may also be obtained by reaction of a free acid with a base such as sodium, potassium or lithium hydroxide, bicarbonate or carbonate, and the like.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

A combination as used herein intends that the individual active ingredients of the compositions are separately formulated for use in combination and can be separately packaged with or without specific dosages. The active ingredients of the combination can be administered concurrently or sequentially.

An “effective amount” is an amount sufficient to effect beneficial or desired results. An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific agent employed, bioavailability of the agent, the route of administration, the age of the animal and its body weight, general health, sex, the diet of the animal, the time of administration, the rate of excretion, the drug combination, and the severity of the particular disorder being treated and form of administration. In general, one will desire to administer an amount of the agent that is effective to achieve a serum level commensurate with the concentrations found to be effective in vivo. These considerations, as well as effective formulations and administration procedures are well known in the art and are described in standard textbooks.

“Therapeutically effective amount” of an agent refers to an amount of the agent that is an amount sufficient to obtain a pharmacological response; or alternatively, is an amount of the agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose and may occur only after administration of a series of doses. Thus, a therapeutically effective amount may be administered in one or more administrations.

In some embodiments, a composition disclosed herein is a cosmetic composition. As used herein, a “cosmetic composition” includes a composition suitable for cleansing or clearing the lungs of irritants when used at a cosmetically effective amount. As used herein, a “cosmetically effective amount” refers to an amount of a compound sufficient to cleanse the lungs of a subject from at least one irritant that is localized in the lungs. In some embodiments, the cosmetic compositions of the present disclosure are delivered by inhalation. In some embodiments, the cosmetic compositions of the present disclosure are delivered by a nebulizer, an atomizer, or a misting machine. In some embodiments, the cosmetic compositions are dry powder compositions. In some embodiments, the cosmetic compositions are liquid compositions.

As used herein, the phrase “derived from” means isolated from, purified from, or engineered from, or any combination thereof.

In some embodiments, the terms “first” “second” “third” “fourth” or similar in a component name are used to distinguish and identify more than one component sharing certain identity in their names. For example, “first oncolytic virus” and “second oncolytic virus” are used to distinguishing two oncolytic viruses.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

As used herein, the term “animal” refers to living multi-cellular vertebrate organisms, a category that includes, for example, mammals and birds. The term “mammal” includes both human and non-human mammals. A preferred subject is one that utilizes lungs to breathe.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human.

In one embodiment, the term “disease” or “disorder” as used herein refers to a pulmonary disease, a status of being diagnosed with a pulmonary disease, a status of being suspect of having a pulmonary disease, or a status of at high risk of having a pulmonary disease.

An agent of the present disclosure can be administered for therapy by any suitable route of administration. It will also be appreciated that the optimal route will vary with the condition and age of the recipient, and the disease being treated.

Administration or treatment in “combination” refers to administering two agents such that their pharmacological effects are manifest at the same time. Combination does not require administration at the same time or substantially the same time, although combination can include such administrations.

As used herein, the term “mucus” generally refers to a usually clear viscid fluid that is secreted by mucous membranes in various tissues of the body, including by the respiratory, gastrointestinal, and reproductive tracts. Mucus moistens, lubricates and protects the tissues from which it is secreted. It comprises mucin macromolecules (including mucus proteins, nucleic acids and carbohydrates), which are the gel-forming constituents of mucus. Mucus proteins include but are not limited to respiratory mucus proteins and digestive tract mucus proteins. The viscoelastic properties of normal mucus are dependent on the concentration, molecular weight, and degree of entanglement between mucin polymers. The term “sputum” generally refers to a mixture of saliva and discharge from the respiratory passages, including mucus. Sputum is typically an expectorated mixture of saliva and mucus (and other discharge from the respiratory tissues). Therefore, mucus is a primary component of sputum, and as such, the presence of excessively viscous mucus results in a sputum which is itself excessively viscous. The present disclosure relates to decreasing the viscosity of the mucus or sputum. The term “liquefaction” refers to the act of becoming more liquid. Therefore, an increase in the liquefaction of mucus or sputum refers to the increase in liquid phase or liquid state of mucus or sputum, as compared to a more solid or viscous phase. In the case of abnormally viscous or excessive mucus associated with disease, the objective is to restore a normal level of mucus viscosity. Hence, liquefaction may also be considered as a reduction in mucus viscosity.

As used herein, a “reducing agent” refers to a substance that reduces a chemical compound usually by donating electrons. As used herein, the phrase “a thiol group” or “a sulfhydryl group,” or “a sulfanyl group” refers to the —SH functional group. As used herein, a “thiol-containing reducing agent” refers to a reducing agent that contains at least one thiol group.

As used herein, the phrase “citric acid” refers to a weak organic acid (C₂H₄O₂) that could exist in an anhydrous or as a monohydrate form. This term also refers to the conjugate base (e.g., a citrate) of citric acid (C₂H₅O(COO)₃³⁻) or to the esters of citric acid.

As used herein, the phrase “carbonic acid” refers to an organic compound with the chemical formula H₂CO₃. This term also refers to a conjugate base of carbonic acid (e.g., bicarbonate), a salt of carbonic acid (e.g., carbonate), or to esters of carbonic acid (e.g., carbonate ester, organic carbonate or organocarbonate).

In some embodiments, N-Acetyl cysteine has the following chemical structure:

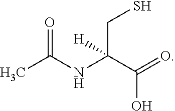

In some embodiments, homocysteine has the following chemical structure:

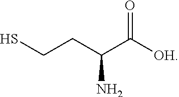

In some embodiments, dihydrolipoic acid has the following chemical structure:

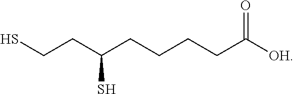 [SMILES: O=C(O)CCCC[C@@H](S)CCS]

In some embodiments, glutathione has the following chemical structure:

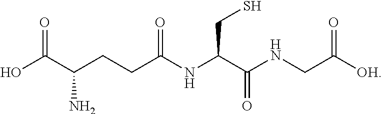 [SMILES: N[C@@H](CCC(=O)N[C@@H](CS)C(=O)NCC(=O)O)C(=O)O]

In some embodiments, coenzyme A has the following chemical structure:

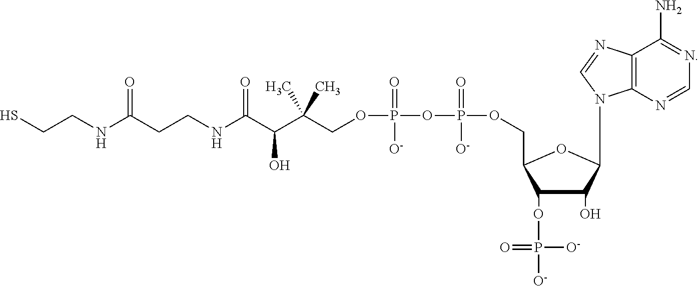

In some embodiments, ergothioneine has the following chemical structure:

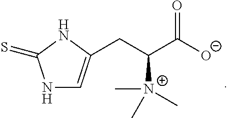

In some embodiments, mycothiol has the following chemical structure:

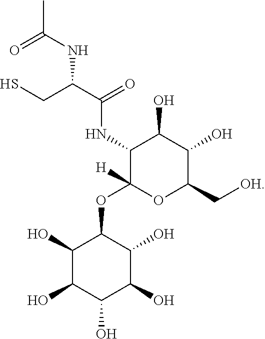 [SMILES: CC(=O)N[C@@H](CS)C(=O)N[C@H]1[C@@H](O[C@@H]2[C@H](O)[C@H](O)[C@@H](O)[C@H](O)[C@H]2O)O[C@H](CO)[C@@H](O)[C@@H]1O]

In some embodiments, bacillithiol has the following chemical structure:

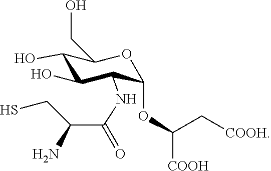

In some embodiments, bicarbonate has the following chemical structure:

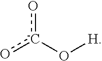

In some embodiments, carbonate has the following chemical structure:

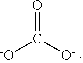

In some embodiments, ascorbic acid has the following chemical structure:

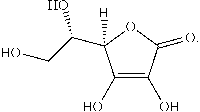 [SMILES: O=C1O[C@H]([C@@H](O)CO)C(O)=C1O]

In some embodiments, Ethylenediaminetetraacetic acid (EDTA) has the following chemical structure:

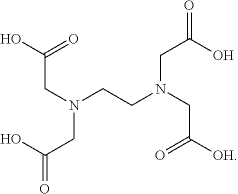 [SMILES: O=C(O)CN(CCN(CC(=O)O)CC(=O)O)CC(=O)O]

In some embodiments, caffeine citrate has the following chemical structure:

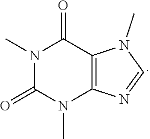

In some embodiments, L-Arginine has the following chemical structure:

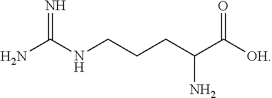 [SMILES: N=C(N)NCCCC(N)C(=O)O]

In some embodiments, Forskolin has the following chemical structure:

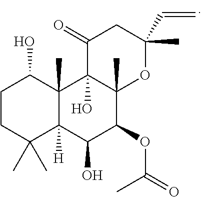

In some embodiments, Theobromine has the following chemical structure:

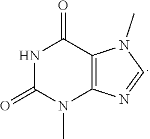

In some embodiments, resolving D1 (RvD1) has the following chemical structure:

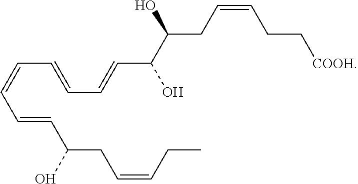 [SMILES: CC/C=C\C[C@H](O)/C=C/C=C\C=C\C=C\[C@@H](O)[C@@H](O)C/C=C\CCC(=O)O]

In some embodiments, resolving E1 (RvE1) has the following chemical structure:

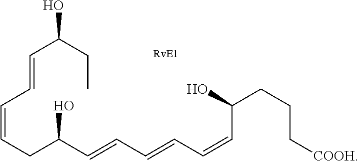 [SMILES: CC[C@H](O)/C=C/C=C\C[C@@H](O)/C=C/C=C/C=C\[C@@H](O)CCCC(=O)O]

In some embodiments, lipoxin A4 (LXA4) has the following chemical structure:

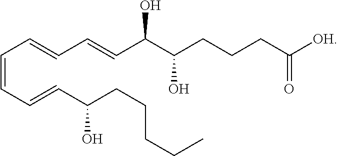 [SMILES: CCCCC[C@H](O)/C=C/C=C\C=C\C=C\[C@@H](O)[C@@H](O)CCCC(=O)O]

In some embodiments, lipoxin B4 (LXB4) has the following chemical structure:

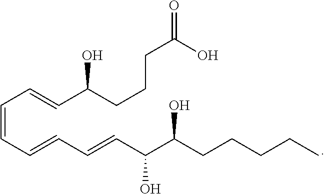 [SMILES: CCCCC[C@H](O)[C@H](O)/C=C/C=C/C=C\C=C\[C@@H](O)CCCC(=O)O]

In some embodiments, protectin D1 (PD1) has the following chemical structure:

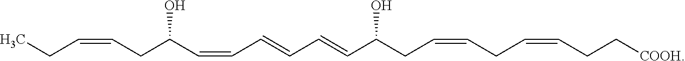 [SMILES: CC/C=C\C[C@H](O)\C=C/C=C/C=C/[C@H](O)C/C=C\C/C=C\CCC(=O)O]

In some embodiments, maresin 1 (MaR1) has the following chemical structure:

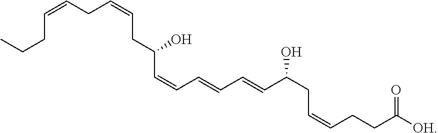 [SMILES: CCC/C=C\C/C=C\C[C@H](O)\C=C/C=C/C=C/[C@H](O)C/C=C\CCC(=O)O]

In some embodiments, maresin 2 (MaR2) has the following chemical structure:

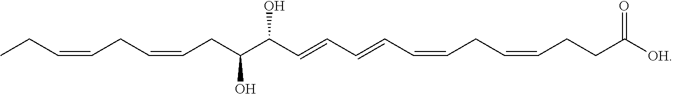 [SMILES: CC/C=C\C/C=C\C[C@H](O)[C@H](O)/C=C/C=C/C=C\C/C=C\CCC(=O)O]

In some embodiments, 17-hydroxy Docosahexaenoic Acid (17-HDHA) has the following chemical structure:

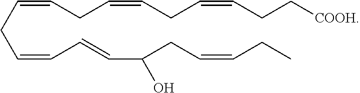 [SMILES: CC/C=C\CC(O)/C=C/C=C\C/C=C\C/C=C\C/C=C\CCC(=O)O]

In some embodiments, 14(S)-hydroxy Docosahexaenoic Acid (14-HDHA) has the following chemical structure:

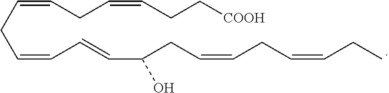 [SMILES: CC/C=C\C/C=C\C[C@H](O)/C=C/C=C\C/C=C\C/C=C\CCC(=O)O]

In some embodiments, 18-HEPE has the following chemical structure:

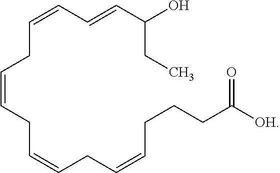 [SMILES: CCC(O)/C=C/C=C\C/C=C\C/C=C\C/C=C\CCCC(=O)O]

As used herein, the term “irritant” or “environmental irritant” or “pollutant” refers to an allergen, bacteria, fungus, prion or other non-viral agent that causes or is associated with pulmonary inflammation, airway hyperreactivity and/or loss of lung function and/or a symptom related thereto. In some embodiments, an irritant inhibits or makes it harder in a subject, normal, efficient breathing or that promotes the production of an undesirable matter (e.g., mucus) that covers a surface of the lungs. In some embodiments, the at least one irritant comprises a cigarette smoke, bacteria, a mold, dust mites, acarids, a pollen, a material from an insect, a material from an animal (e.g., cats, dogs, rabbits, mice, rats, hamsters, guinea pigs, and birds) and animal dander, fungi, an air pollutant (e.g., smoke, smog, microparticulate matter), an irritant aerosol, a fume, a noxious chemical, or combinations thereof.

As used herein, the term “stabilizer” refers to an excipient utilized to control interparticle cohesive forces between particles. In some embodiments, a stabilizer maintains physicochemical properties of the powder including aerosol particle size. Stabilizers are typically hydrophobic and include, but are not limited to, magnesium stearate, leucine, and long-chain saturated phospholipids (e.g., dipalmitoylphosphatidylcholine). In some embodiments, the stabilizer comprises leucine, lecithin, magnesium stereate, trileucine, isoleucine, glycine, lactose, mannitol, trehalose, erythritol, sorbitol, or combinations thereof.

### Modes for Carrying Out the Disclosure

The present disclosure provides compositions that are useful for alleviating one or more negative effects of a pulmonary or airway disorder and for enhancing clearance of pollutants, microparticulate matter, or other irritants from the lungs of a subject. The present disclosure also provides methods using the disclosed compositions.

### Compositions

An aspect of the disclosure is directed to a composition comprising: (a) a thiol-containing reducing agent; (b) citric acid or a physiologically acceptable salt thereof; (c) carbonic acid or a physiologically acceptable salt thereof; and (d) a stabilizer.

In some embodiments, the composition comprises (a) 40%-80% (w/w) (e.g., 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80% (w/w), or any value therebetween) of a thiol-containing reducing agent; (b) 8%-20% (w/w) (e.g., 8, 9, 10, 11, 12, 13, 14, 15, 16, 17, 18, 19, 20% (w/w), or any value therebetween) of citric acid or a physiologically acceptable salt thereof; (c) 8%-20% (w/w) (e.g., 8, 9, 10, 11, 12, 13, 14, 15, 16, 17, 18, 19, 20% (w/w), or any value therebetween) of carbonic acid or a physiologically acceptable salt thereof; and (d) 3%-6% (w/w) (e.g., 3, 3.5, 4, 4.5, 5, 5.5, 6% (w/w), or any value therebetween) of a stabilizer.

In some embodiments, the composition comprises (a) 50%-70% (w/w) (e.g., 50, 51, 52, 53, 54, 55, 56, 57, 58, 59, 60, 61, 62, 63, 64, 65, 66, 67, 68, 69, 70% (w/w), or any value therebetween) of a thiol-containing reducing agent; (b) 10%-15% (w/w) (10, 11, 12, 13, 14, 15% (w/w), or any value therebetween) of citric acid or a physiologically acceptable salt thereof; (c) 10%-15% (w/w) of carbonic acid or a physiologically acceptable salt thereof; and (d) 3.5%-5% (w/w) (e.g., 3.5, 4, 4.5, 5% (w/w), or any value therebetween) of a stabilizer.

In some embodiments, the composition comprises (a) 60%-65% (w/w) (e.g., 60, 61, 62, 63, 64, 65% (w/w) of a thiol-containing reducing agent; (b) 12%-15% (w/w) (e.g., 12, 13, 14, 15% (w/w), or any value therebetween) of citric acid or a physiologically acceptable salt thereof; (c) 12%-15% (w/w) (e.g., 12, 13, 14, 15% (w/w), or any value therebetween) of carbonic acid or a physiologically acceptable salt thereof; and (d) 4%-4.5% (w/w) (e.g., 4, 4.3, 4.5% (w/w), or any value therebetween) of a stabilizer.

In some embodiments, the composition comprises (a) about 60% (w/w) of a thiol-containing reducing agent; (b) about 15% (w/w) of citric acid or a physiologically acceptable salt thereof; (c) about 13% (w/w) of carbonic acid or a physiologically acceptable salt thereof; and (d) about 4% (w/w) of a stabilizer.

In some embodiments, the thiol-containing reducing agent comprises cysteine, N-Acetyl cysteine, homocysteine, dihydrolipoic acid, glutathione, coenzyme A, ergothioneine, mycothiol, bacillithiol, or combinations thereof.

In some embodiments, the stabilizer comprises leucine, lecithin, magnesium stereate, trileucine, isoleucine, glycine, lactose, mannitol, trehalose, erythritol, sorbitol, or combinations thereof.

In some embodiments, the molar ratio of (a):(b):(c):(d) ranges from about 0.5-1:0.5-1:0.5-1:0.5-1, or any combination of values therebetween.

In some embodiments, the molar ratio of (a):(b):(c):(d) is about 0.5-0.7:0.5-0.7:0.5-0.7:0.7-1. In some embodiments, the molar ratio of (a):(b):(c):(d) is about 0.5-0.7:0.5-0.7:0.7-1:0.7-1. In some embodiments, the molar ratio of (a):(b):(c):(d) is about 0.5-0.7:0.7-1:0.7-1:0.7-1. In some embodiments, the molar ratio of (a):(b):(c):(d) is about 0.7-1:0.7-1:0.7-1:0.7-1.

In some embodiments, the molar ratio of (a):(b):(c):(d) is about 0.7-1:0.5-0.7:0.5-0.7:0.5-0.7. In some embodiments, the molar ratio of (a):(b):(c):(d) is about 0.7-1:0.7-1:0.5-0.7:0.5-0.7. In some embodiments, the molar ratio of (a):(b):(c):(d) is about 0.7-1:0.7-1:0.7-1:0.5-0.7.

In some embodiments, the pH of the composition ranges from about 5.5 to about 10 (e.g., about pH 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.2, 9.3, 9.4, 9.5, 9.6, 9.7, 9.8, 9.9, or 10) about 5.5 to about 8 (e.g., about pH 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, or 8), about 6 to about 10 (e.g., about pH 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.2, 9.3, 9.4, 9.5, 9.6, 9.7, 9.8, 9.9, or 10), or about 6 to about 8 (e.g., about pH 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, or 8).

In some embodiments, the composition comprises an aqueous solution. In some embodiments, the aqueous solution is nebulized using a liquid nebulizer. In some embodiments, the nebulizer used in the present disclosure is a jet nebulizer, an ultrasonic nebulizer, a pulsating membrane nebulizer, a nebulizer comprising a vibrating mesh or plate with multiple apertures, or a nebulizer comprising a vibration generator and an aqueous chamber.

In some embodiments, the composition comprises a dry powder. In some embodiments, the composition is packaged into an inhaler. In some embodiments, an inhaler is a passive dry powder inhaler (DPI), such as a Plastiape RSO1 monodose DPI. In some embodiments, in a dry powder inhaler, dry powder is stored in a reservoir and is delivered to the lungs by inhalation without the use of propellants.

In some embodiments, an inhaler is a single-dose DPI, such as a DoseOne™, Spinhaler, Rotohaler®, Aerolizer®, or Handihaler. In some embodiments, an inhaler is a multidose DPI, such as a Plastiape RSO2, Turbuhaler®, Twisthaler™, Diskhaler®, Diskus®, or Ellipta™. In some embodiments, an inhaler is a plurimonodose DPI for the concurrent delivery of single doses of multiple medications, such as a Plastiape RSO4 plurimonodose DPI. In some embodiments, dry powder inhalers have medication stored in an internal reservoir, and medication is delivered by inhalation with or without the use of propellants.

In some embodiments, the composition is suitable for administration by inhalation.

In some embodiments, the composition further comprises at least one ingredient selected from Adhatoda vasica Nees extract, Albizzia lebbeck (Sareesha_rakat) extract, Alstonia scholaris extract, Artemisia caerulescens extract, Belamcanda chinensis extract, Benincasa hispida extract, Cissampelos sympodialis extract, Clerodendron serratum extract, Coleus forskohlii extract, Elaeocarpus spharicus extract, Galphimia glauca extract, Gardenia latifolia extract, Gingko biloba extract, Mikania glomerata extract, Lepidium sativum extract, Ocimum sanctum extract, Passiflora incarnata extract, Pavetta crassipes extract, Picrorrhiza kurroa extract, Sarcostemma brevistigma extract, Tephrosia purpurea extract, Tylophora indica extract, Vitex negundo extract, Rosmarinus officinalis extract, Ephedra sinica extract, and Gleditsia sinensis Lam extract.

In some embodiments, the composition further comprises at least one additional ingredient selected from bicarbonate, Ascorbic acid, EDTA, N-acetylcysteine, Caffeine citrate, L-Arginine, Forskolin, Theobromine, resolving D1 (RvD1), resolving E1 (RvE1), lipoxin A4 (LXA4), lipoxin B4 (LXB4), protectin D1 (PD1), maresin 1 (MaR1), maresin 2 (MaR2), 17-HDHA, 14-HDHA, ellagic acid, curcumin, 18-HEPE, or a combination thereof. In some embodiments, the at least one additional ingredient is present in the composition between 0.1% and 10%, or any amount therebetween.

In some embodiments, the composition comprises a lyophilized dry powder. In some embodiments, the lyophilized dry powder is produced by spray drying or by air jet milling (aka “top-down approach”).

Another aspect of the disclosure is directed to a dry powder composition suitable for administration to a human subject by inhalation comprising (a) cysteine, (b) citric acid or a physiologically acceptable salt thereof, (c) carbonic acid or a physiologically acceptable salt thereof, and (d) leucine.

In some embodiments, the dry powder composition comprises (a) 40%-80% (w/w) (e.g., 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80% (w/w), or any value therebetween) of a thiol-containing reducing agent; (b) 8%-20% (w/w) (e.g., 8, 9, 10, 11, 12, 13, 14, 15, 16, 17, 18, 19, 20% (w/w), or any value therebetween) of citric acid or a physiologically acceptable salt thereof; (c) 8%-20% (w/w) (e.g., 8, 9, 10, 11, 12, 13, 14, 15, 16, 17, 18, 19, 20% (w/w), or any value therebetween) of carbonic acid or a physiologically acceptable salt thereof; and (d) 3%-6% (w/w) (e.g., 3, 3.5, 4, 4.5, 5, 5.5, 6% (w/w), or any value therebetween) of a stabilizer.

In some embodiments, the dry powder composition comprises (a) 50%-70% (w/w) (e.g., 50, 51, 52, 53, 54, 55, 56, 57, 58, 59, 60, 61, 62, 63, 64, 65, 66, 67, 68, 69, 70% (w/w), or any value therebetween) of a thiol-containing reducing agent; (b) 10%-15% (w/w) (10, 11, 12, 13, 14, 15% (w/w), or any value therebetween) of citric acid or a physiologically acceptable salt thereof; (c) 10%-15% (w/w) of carbonic acid or a physiologically acceptable salt thereof; and (d) 3.5%-5% (w/w) (e.g., 3.5, 4, 4.5, 5% (w/w), or any value therebetween) of a stabilizer.

In some embodiments, the dry powder composition comprises (a) 60%-65% (w/w) (e.g., 60, 61, 62, 63, 64, 65% (w/w) of a thiol-containing reducing agent; (b) 12%-15% (w/w) (e.g., 12, 13, 14, 15% (w/w), or any value therebetween) of citric acid or a physiologically acceptable salt thereof; (c) 12%-15% (w/w) (e.g., 12, 13, 14, 15% (w/w), or any value therebetween) of carbonic acid or a physiologically acceptable salt thereof; and (d) 4%-4.5% (w/w) (e.g., 4, 4.3, 4.5% (w/w), or any value therebetween) of a stabilizer.

In some embodiments, the dry powder composition comprises (a) about 60% (w/w) of a thiol-containing reducing agent; (b) about 15% (w/w) of citric acid or a physiologically acceptable salt thereof; (c) about 13% (w/w) of carbonic acid or a physiologically acceptable salt thereof; and (d) about 4% (w/w) of a stabilizer.

In some embodiments, the molar ratio of (a):(b):(c):(d) ranges from about 0.5-1:0.5-1:0.5-1:0.5-1, or any combination of values therebetween.

In some embodiments, the molar ratio of (a):(b):(c):(d) is about 0.5-0.7:0.5-0.7:0.5-0.7:0.7-1. In some embodiments, the molar ratio of (a):(b):(c):(d) is about 0.5-0.7:0.5-0.7:0.7-1:0.7-1. In some embodiments, the molar ratio of (a):(b):(c):(d) is about 0.5-0.7:0.7-1:0.7-1:0.7-1. In some embodiments, the molar ratio of (a):(b):(c):(d) is about 0.7-1:0.7-1:0.7-1:0.7-1.

In some embodiments, the molar ratio of (a):(b):(c):(d) is about 0.7-1:0.5-0.7:0.5-0.7:0.5-0.7. In some embodiments, the molar ratio of (a):(b):(c):(d) is about 0.7-1:0.7-1:0.5-0.7:0.5-0.7.

In some embodiments, the molar ratio of (a):(b):(c):(d) is about 0.7-1:0.7-1:0.7-1:0.5-0.7.

In some embodiments, the pH of the composition ranges from about 5.5 to about 10 (e.g., about pH 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.2, 9.3, 9.4, 9.5, 9.6, 9.7, 9.8, 9.9, or 10) about 5.5 to about 8 (e.g., about pH 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, or 8), about 6 to about 10 (e.g., about pH 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.2, 9.3, 9.4, 9.5, 9.6, 9.7, 9.8, 9.9, or 10), or about 6 to about 8 (e.g., about pH 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, or 8).

In some embodiments, the dry powder composition further comprises at least one ingredient selected from Adhatoda vasica Nees extract, Albizzia lebbeck (Sareesha_rakat) extract, Alstonia scholaris extract, Artemisia caerulescens extract, Belamcanda chinensis extract, Benincasa hispida extract, Cissampelos sympodialis extract, Clerodendron serratum extract, Coleus forskohlii extract, Elaeocarpus spharicus extract, Galphimia glauca extract, Gardenia latifolia extract, Gingko biloba extract, Mikania glomerata extract, Lepidium sativum extract, Ocimum sanctum extract, Passiflora incarnata extract, Pavetta crassipes extract, Picrorrhiza kurroa extract, Sarcostemma brevistigma extract, Tephrosia purpurea extract, Tylophora indica extract, Vitex negundo extract, Rosmarinus officinalis extract, Ephedra sinica extract, and Gleditsia sinensis Lam extract.

In some embodiments, the dry powder composition further comprises at least one additional ingredient selected from bicarbonate, Ascorbic acid, EDTA, N-acetylcysteine, Caffeine citrate, L-Arginine, Forskolin, Theobromine, resolving D1 (RvD1), resolving E1 (RvE1), lipoxin A4 (LXA4), lipoxin B4 (LXB4), protectin D1 (PD1), maresin 1 (MaR1), maresin 2 (MaR2), 17-HDHA, 14-HDHA, ellagic acid, curcumin, 18-HEPE, or a combination thereof. In some embodiments, the at least one additional ingredient is present in the composition between 0.1% and 10%, or any amount therebetween.

In some embodiments, the dry powder composition is packaged into an inhaler. In some embodiments, the inhaler is a dry powder inhaler (DPI).

### Methods

An aspect of the disclosure is directed to methods that use the compositions described herein.

Another aspect of the disclosure is directed to a method of alleviating one or more negative effects of a pulmonary or airway disorder comprising administering to a subject in need thereof a composition arising from a combination of a thiol-containing reducing agent, citric acid or a physiologically acceptable salt thereof, carbonic acid or a physiologically acceptable salt thereof, and a stabilizer.

Another aspect of the disclosure is directed to a method of enhancing clearance of pollutants, microparticulate matter, or other irritants from the lungs of a subject comprising administering to a subject in need thereof a composition arising from a combination of a thiol-containing reducing agent, citric acid or a physiologically acceptable salt thereof, carbonic acid or a physiologically acceptable salt thereof, and a stabilizer.

Another aspect of the disclosure is directed to a method of cleansing at least a portion of the lungs of a subject comprising administering to a subject in need thereof a composition arising from a combination of a thiol-containing reducing agent, citric acid or a physiologically acceptable salt thereof, carbonic acid or a physiologically acceptable salt thereof, and a stabilizer.

In some embodiments, the pulmonary or airway disorder is selected from chronic inflammatory lung disease, pulmonary fibrosis, pulmonary vasculitis, pulmonary sarcoidosis, inflammation and/or infection associated with lung transplantation, acute or lung rejection and/or dysfunction, pulmonary artery hypertension, bronchitis, sinusitis, asthma, cystic fibrosis, bronchiectasis, bacterial infection, fungal infection, parasite infection, viral infection, chronic obstructive pulmonary disease (COPD), bronchiolitis obliterans syndrome (BOS), primary ciliary dyskinesia (PCD), alveolar proteinosis, idiopathic pulmonary fibrosis, eosinophilic pneumonia, eosinophilic bronchitis, acute respiratory distress syndrome (ARDS), inflammation and/or infection associated with mechanical ventilation, ventilator-associated pneumonia, asbestos-related airway disorder or disease, dust-related airway disorder or disease, silicosis, and radiation or chemical agent-related airway disease or disorder, and any combination thereof.

Another aspect of the disclosure is directed to a method of enhancing clearance of pollutants, microparticulate matter, or other irritants from the lungs of a subject comprising administering to a subject in need thereof a composition arising from a combination of a thiol-containing reducing agent, citric acid or a physiologically acceptable salt thereof, carbonic acid or a physiologically acceptable salt thereof, and a stabilizer.

Another aspect of the disclosure is directed to a method of cleansing at least a portion of the lungs of a subject comprising administering to at least a portion of the lungs of a subject in need thereof a composition arising from a combination of a thiol-containing reducing agent, citric acid or a physiologically acceptable salt thereof, carbonic acid or a physiologically acceptable salt thereof, and a stabilizer.

In some embodiments, at least a portion of the lungs is cleansed of at least one irritant upon using the methods of the present disclosure. In some embodiments, the at least one irritant comprises a cigarette smoke, bacteria, a mold, dust mites, acarids, a pollen, a material from an insect, a material from an animal (e.g., cats, dogs, rabbits, mice, rats, hamsters, guinea pigs, and birds) and animal dander, fungi, an air pollutant (e.g., smoke, smog, microparticulate matter), an irritant aerosol, a fume, a noxious chemical, or combinations thereof.

In some embodiments, the composition includes/comprises a physiologically acceptable carrier.

In some embodiments, the composition is a dry powder administered through inhalation.

In some embodiments, the composition is administered at least once a day, twice a day, three times a day, or as needed.

In some embodiments, the composition is a dry powder administered through inhalation.

In some embodiments, the composition is administered by a dry powder inhaler (DPI).

In some embodiments, the combination excludes divalent cations.

In some embodiments, the thiol-containing reducing agent comprises cysteine, N-Acetyl cysteine, homocysteine, dihydrolipoic acid, glutathione, coenzyme A, ergothioneine, mycothiol, bacillithiol, or combinations thereof.

In some embodiments, the stabilizer comprises leucine, lecithin, magnesium stereate, trileucine, isoleucine, glycine, lactose, mannitol, trehalose, erythritol, sorbitol, or combinations thereof.

In some embodiments, the composition further comprises at least one ingredient selected from Adhatoda vasica Nees extract, Albizzia lebbeck (Sareesha_rakat) extract, Alstonia scholaris extract, Artemisia caerulescens extract, Belamcanda chinensis extract, Benincasa hispida extract, Cissampelos sympodialis extract, Clerodendron serratum extract, Coleus forskohlii extract, Elaeocarpus spharicus extract, Galphimia glauca extract, Gardenia latifolia extract, Gingko biloba extract, Mikania glomerata extract, Lepidium sativum extract, Ocimum sanctum extract, Passiflora incarnata extract, Pavetta crassipes extract, Picrorrhiza kurroa extract, Sarcostemma brevistigma extract, Tephrosia purpurea extract, Tylophora indica extract, Vitex negundo extract, Rosmarinus officinalis extract, Ephedra sinica extract, and Gleditsia sinensis Lam extract.

In some embodiments, the composition further comprises at least one ingredient selected from bicarbonate, Ascorbic acid, EDTA, N-acetylcysteine, Caffeine citrate, L-Arginine, Forskolin, Theobromine, resolving D1 (RvD1), resolving E1 (RvE1), lipoxin A4 (LXA4), lipoxin B4 (LXB4), protectin D1 (PD1), maresin 1 (MaR1), maresin 2 (MaR2), 17-HDHA, 14-HDHA, ellagic acid, curcumin, 18-HEPE, or a combination thereof.

In some embodiments, the composition comprises a lyophilized dry powder. In some embodiments, the lyophilized dry powder is produced by spray drying or by air jet milling (aka. “top-down approach”).

### EXAMPLES

### Example 1: Materials and Methods

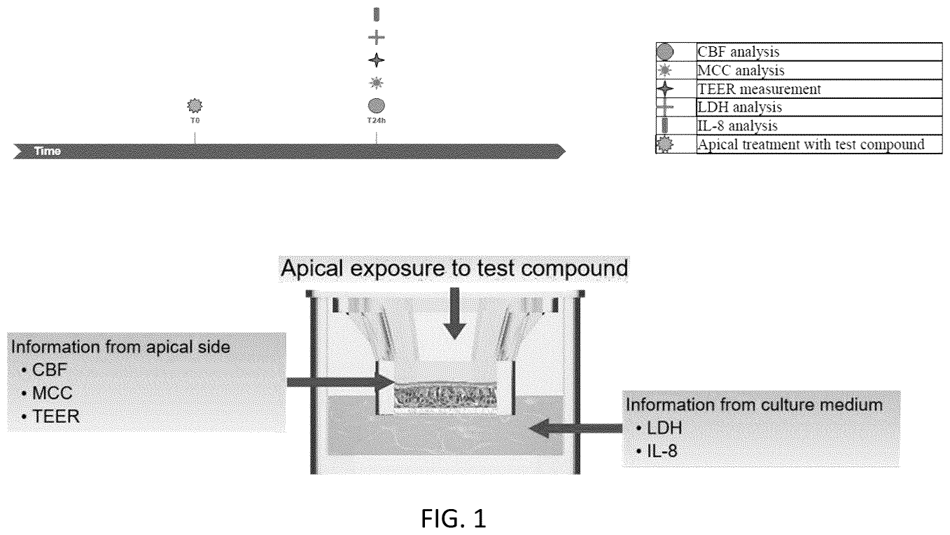

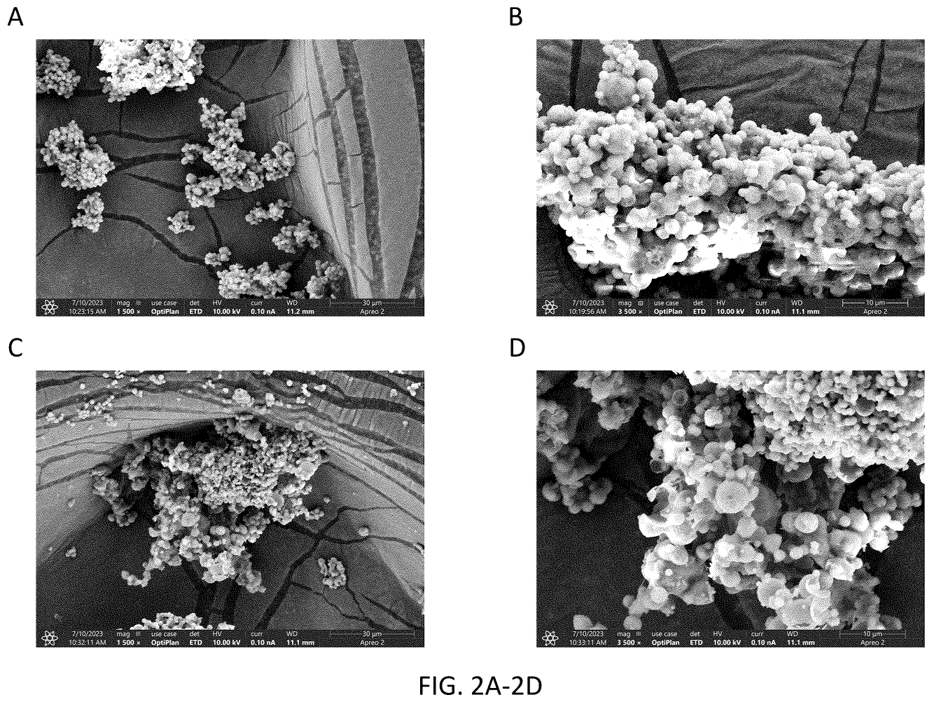

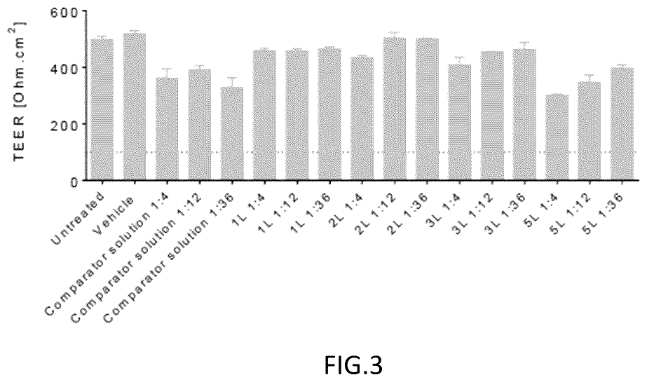

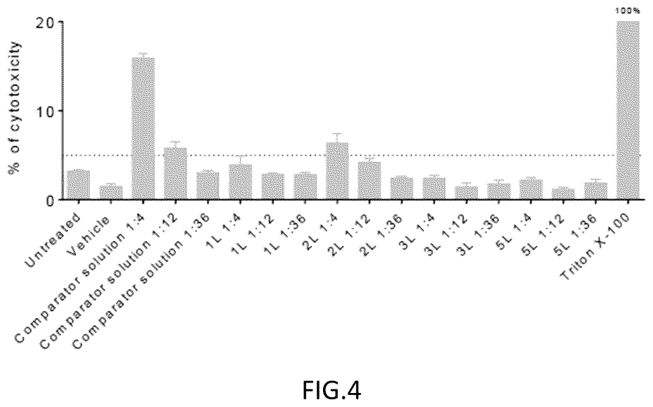

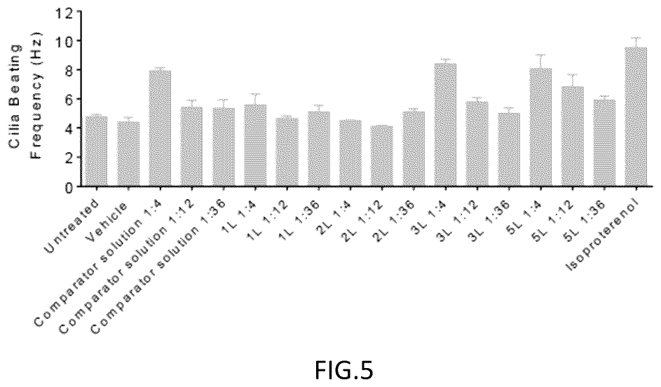

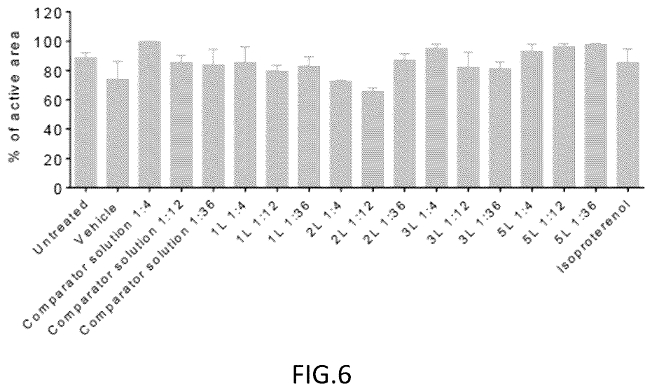

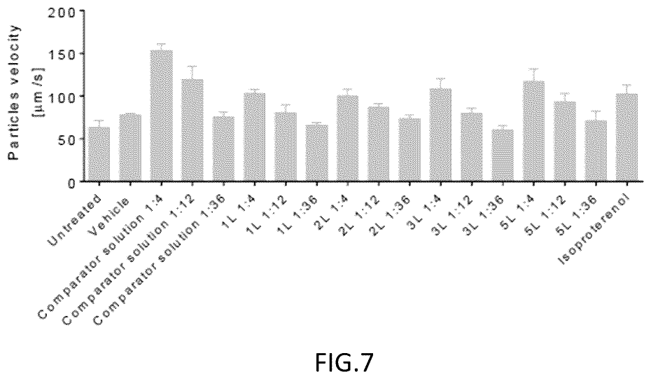

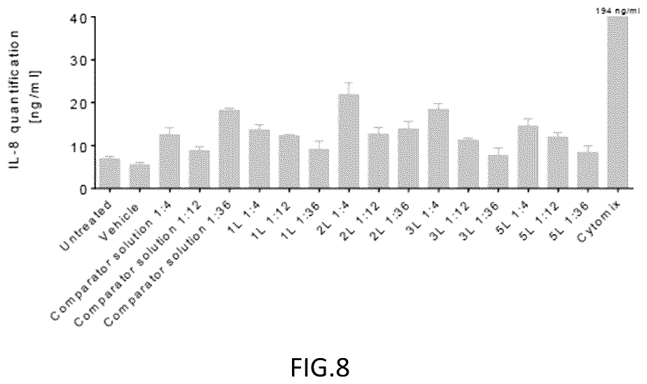

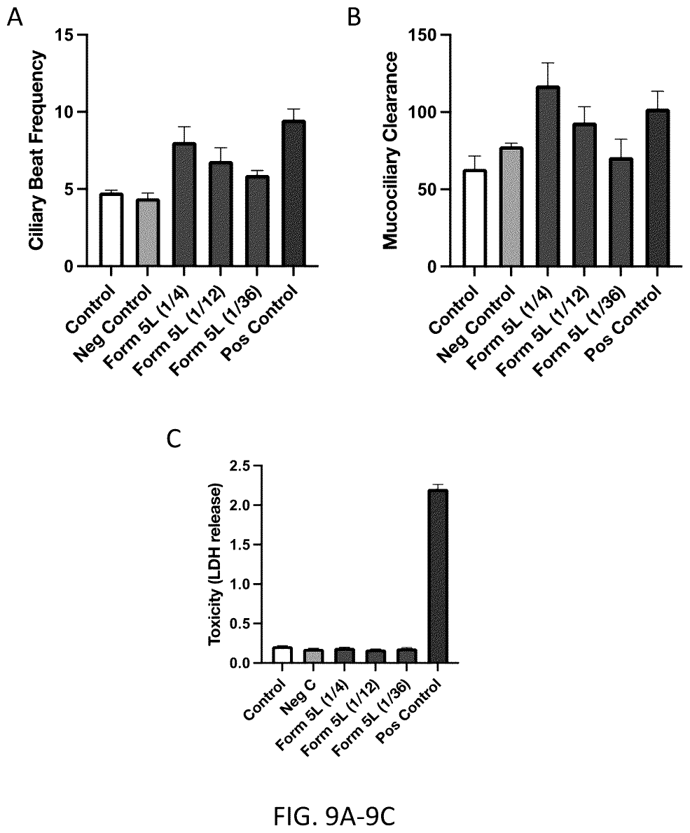

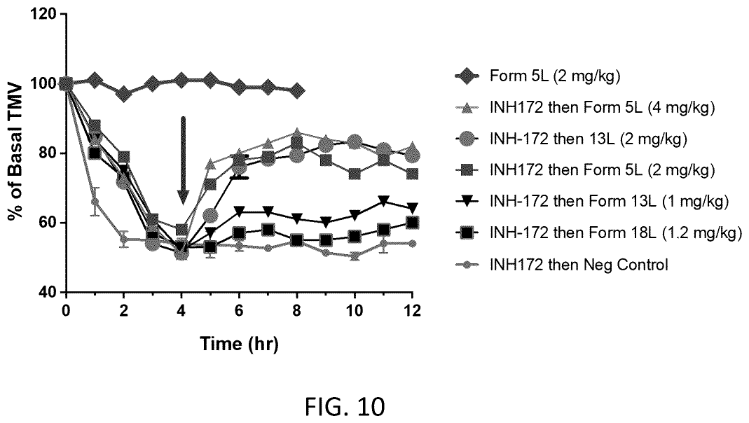

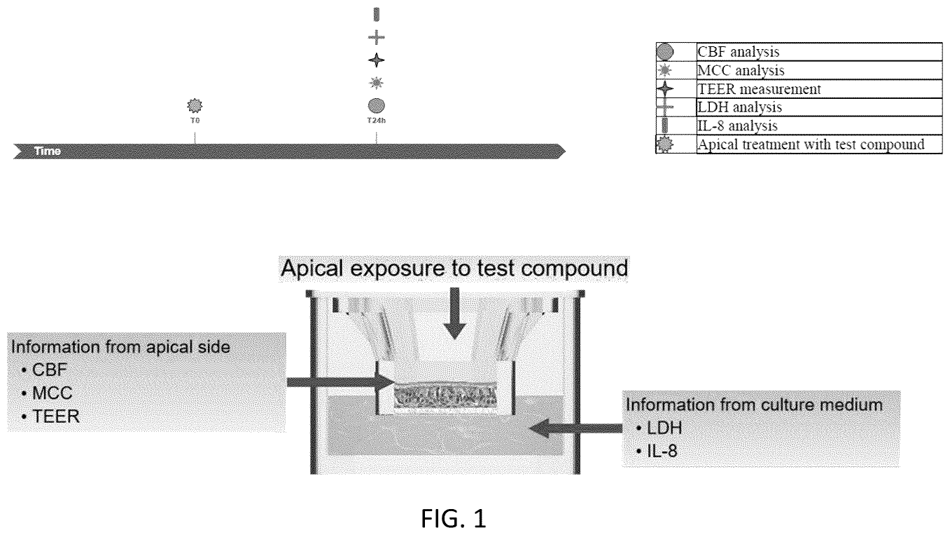

Testing strategy. Several endpoints are evaluated as shown in FIG. 1. For instance, the following endpoints are measured: Global markers of toxicity (e.g., Cytotoxicity: Lactate dehydrogenase (LDH) assay), Tissue integrity monitoring: (e.g., Trans-epithelial electrical resistance (TEER) measurement); Effect on cilia (e.g., Mucus motion: Mucociliary Clearance (MCC), Cilia motion: Cilia Beating Frequency (CBF)); and Effect on cytokines and AMP release (e.g., Basal IL-8: ELISA).

Sheep Mucociliary Clearance (MCC). On the day of the experiment, the amount of the test agent reflecting the molar concentration to be studied is dissolved in 7 ml of normal saline (0.9% NaCl). The solution is then filtered through a 0.22-μm nonpyrogenic filter (CoStar, Cambridge, MA), and 4 ml of this solution were used for aerosolization.

Animal preparation. Adult ewes, 25-35 kg in weight, are restrained in an upright position in a specialized body harness adapted to a modified shopping cart. The animals' heads are immobilized, and local anesthesia of the nasal passage is induced with 2% lidocaine. The animals are then nasally intubated with a 7.5-mm-ID endotracheal tube (ETT) (Mallinckrodt Medical, St. Louis, MO). The cuff of the ETT is placed just below the vocal cords, and its position was verified by a flexible bronchoscope. After intubation, the animals are allowed to equilibrate for a period of ˜20 min before MCC measurements began.

Measurement of MCC. Aerosols of ^{99m}Tc-HSA are generated by a Raindrop Nebulizer (Nellcor Puritan Bennett, Pleasanton, CA), which produces a droplet with a median aerodynamic diameter of 3.6 m. The nebulizer is connected to a dosimetry system consisting of a solenoid valve and a source of compressed air (20 psi). The output of the nebulizer is directed into a plastic T piece, one end of which was connected to the sheep's ETT and the other end to a piston respirator (Harvard Apparatus, South Natick, MA). The system is activated for 1 s at the onset of the respirator's inspiratory cycle. The respirator is set at a tidal volume of 500 ml, an inspiratory-to-expiratory ratio of 1:1, and at a rate of 20 breaths/min to maximize central airway deposition. The sheep breathe the radiolabeled aerosol for 5 min. A gamma camera (Dyna Cam, Picker, Nothford, CT) is used to measure the clearance of ^{99m}Tc-HSA from the airways. The gamma camera is positioned above the animal's back, with the sheep in its natural upright position in the cart, so that the field of image is perpendicular to the animal's spinal cord. External radiolabeled markers are placed on the sheep to facilitate proper alignment under the gamma camera. All deposition images are stored in a computer integrated with the gamma camera. A region of interest is traced over the image corresponding to the right lung of the sheep, and the counts are recorded. The counts are corrected for decay and expressed as percentage of radioactivity present in the initial baseline image. The left lung is excluded from analysis because its outlines are superimposed over the stomach, and counts can be affected by swallowed radiolabeled mucus.

For the MCC studies, a baseline deposition image is obtained immediately after radioaerosol administration. After acquisition of the baseline image, either 0.9% normal saline (control), or test formulations aerosolized from a 4-ml volume by using the Pari LC JetPlus nebulizer to free-breathing sheep. The nebulizer is driven by compressed air with a flow of 8 l/min. The time to deliver the solution is 10-12 min. On the completion of drug administration, the animal is immediately extubated. This was done to prevent false elevations in counts caused by aspiration of excess radiolabeled mucus from the ETT. Serial measurements of the radiolabeled material present in the lungs are obtained over a 2-h period at 5-min intervals for the first hour and then every 15 min for the next hour. A washout period of at least 7 days (half-life of ^{99m}Tc=6 h) separated studies with the different agents.

Test Epithelia Studies. Air-liquid interface” (“ALI”) Cultures are grown following published methods. For example, human bronchial epithelial cells are first expanded in LHC9/RPMI 1640 (1:1) serum-free medium (EUROCLONE®) and then seeded on porous supports (Snapwell™, Corning® Costar®) at 5×10⁵ cells/cm². Cells are induced to differentiate at the “air-liquid interface” (ALI) in LHC9/Ham's F12 (1:1, v:v) supplemented with 2% Ultroser™ G serum substitute (provided by the FFC facility), 2 mM L-glutammine, 100 U/mL penicillin and 100 g/mL streptomycin (all from Euroclone®). Supports are coated with rat-tail collagen diluted 1/100. All cell lines were cultured at 37° C. under 5% CO₂.

In the studies described herein, test epithelia (MucilAir™, Epithelix) are reconstituted from cells isolated from a healthy bronchial donor. The formulation is exposed apically to mimic deposition on the nasal mucosa via inhalation.

The treatments texted in the test epithelia studies are in Table 1.

TABLE 1
Treatments in the test epithelia studies. Each compound
was tested at three dilutions. Each dilution had 5 replicates.
The controls (positive and negative) had 3 replicates.
Each experiment was repeated three times.

| Treatment | Concentration | Dilution |
|---|---|---|
| 1 L | 92.67 mg/ml | 1:4 |
| | | 1:12 |
| | | 1:36 |
| 2 | 67.01 mg/ml | 1:4 |
| | | 1:12 |
| | | 1:36 |
| 3 | 37.62 mg/ml | 1:4 |
| | | 1:12 |
| | | 1:36 |
| 5 L | 93.41 mg/ml | 1:4 |
| | | 1:12 |
| | | 1:36 |
| Comparator solution | | 1:4 |
| | | 1:12 |
| | | 1:36 |
| 0.9% NaCl | | |
| Positive Control | | |

In the studies described herein, negative controls are untreated and vehicle control (0.95 NaCl). The vehicle control is a saline solution apically exposed to the epithelia. The comparator solution is a solution comprised of 84.0 mg/mL sodium bicarbonate, 150.0 mg/mL glutathione and 70.4 mg/mL ascorbic acid.

In the studies described herein, positive controls depend on experiment. For example: Isoproterenol 100 μM in culture medium; CBF activator applied in basolateral compartment (700 l) for 2 hours (CBF positive control); Isoproterenol 100 μM in culture medium (MCC positive control); Cytomix LPS 0.2 mg/ml+TNF-α 0.5 g/ml+1% FBS in culture medium, for inflammation applied in basolateral compartment (500 l) for 24 hours (IL-8 positive control); or Triton X-100, 10% in culture medium, for toxicity applied in apical compartment (100 μl) for 24 hours (cytotoxicity positive control).

Timeline of Epithelia Experiments. At time point 0, epithelia are treated with the test formulation or control using an apical mode of administration. Test formulations or control were exposed at a quantity of 10 μL, at the concentrations in Table 1. Twenty-four hours after treatment, CBF analysis, MCC analysis, TEER measurement, LDH analysis, and IL-8 analysis are performed on the animals. See FIG. 1.

Epithelial MCC. MCC is monitored using a Sony® XCD-U100CR camera connected to an Axiovert 200M microscope (Zeiss®) with a 5× objective. Polystyrene microbeads (30 μM diameter—Sigma®, 84315) are added on the apical surface of the epithelia. Microbead movements are video tracked at 2 frames per second for 30 images for room temperature. Three movies are taken per insert. Average bead movement velocity (μM/sec) are calculated with ImageProPlus 6.0 software to determine MCC.

Trans-epithelial electrical resistance (TEER). To assess differentiation in primary cultures, measurement of TEER was performed using chopstick electrodes coupled with an ohmmeter (Millicell-ERS, Millipore, Billerica, Massachusetts, USA), after the addition of 200 to 500 μL medium (or 0.9% NaCl in the controls) to the apical surface of cultures. Epithelial differentiation was achieved for TEER values of at least 400-500 Ω/cm².

Resistance was measured with an EVOMX® volt-ohm-meter (World Precision Instruments UK, Stevenage) for each condition. Resistance values (Ω) were converted to TEER (2.cm²) using the following formula:

$$TEER ( Ω . {cm}^{2} ) = ( resistance value ( Ω ) - 100 ( Ω ) ) \times 0.33 ( {cm}^{2} ) , where 100 Ω is the resistance of the membrane and 0.33 {cm}^{2} is the total surface of the epithelium .$$

The change of TEER reflects the integrity/state of epithelia. For example, if holes were present or if cellular junction were broken, the TEER values would be generally below 100 Ω·cm². In contrast, when epithelia are not damaged, the TEER values are typically comprised between 200 to 600 Ω·cm². A notable decrease of the TEER value (but>100 Ω·cm²) could be observed in certain cases. This change generally reflects an activation of the ion channels. A drastic increase of the TEER value reflects a blockage of the ion channel activity or a destruction of the ciliated cells.

Cytotoxicity (LDH). Cytotoxicity of the formulation is measured by quantifying the lactate dehydrogenase released in the medium by dead cells (Cytotoxicity LDH Assay Kit-WST (Dojindo, réf.CK12-20)). LDH is a stable cytoplasmic enzyme that is rapidly released into culture medium upon rupture of the plasma membrane.

100 μL basolateral medium collected at each time-point is incubated with the reaction mixture of the LDH Assay Kit-WST. Released LDH is quantified measuring the absorbance of each sample at 490 nm with a microplate reader. Cytotoxicity is calculated using the following equation, where A is absorbance values:

$$Cytotoxicity ( \% ) = ( A ( exp . value ) - A ( lowcontrol ) / A ( highcontol ) - A ( lowcontrol ) ) * 100$$

In the LDH assays, 10% Triton X-100 is the positive control, because it causes massive LDH release. The value obtained by 10% Triton X-100 corresponds to 100% cytotoxicity. The negative controls show a LDH of <5%, which is due to physiological cell turnover in the test epithelia.

Ciliary beat frequency (CBF). In some embodiments, CBF is analyzed with Sisson-Ammons Video Analysis (SAVA) software (Sisson et al., Journal of microscopy 211.2 (2003): 103-111) using an inverted phase contrast microscope (Zeiss®, Germany). In some embodiments, CBF is analyzed with Epithelix software (Cilia-X). Live cells are visualized on a heated stage at 100× magnification (10× ocular, 10× objective), focused to give a view of the cells and cilia. In some embodiments, two videos are recorded per well at 120 frames per second with a total of 512 frames per video. In other embodiments, the CBF is expressed as Hz 256, wherein movies are captured at a high frequency rate of 125 frames per second.

The software is used to calculate the percentage active area within the region of interest and CBF for all samples.

Cytokine release (IL-8). The release of IL-8 is measured by ELISA technique, according to the manufacturer's instructions. Lyophilized standard is reconstituted, aliquoted and stored at −80° C. Each ELISA plate contained a standard curve. Medium samples are diluted with the appropriate assay diluent at the appropriate dilution rate, washing steps are performed with an automatic microplate washer and absorbance is measured at 450 nm.

### Example 2: Results

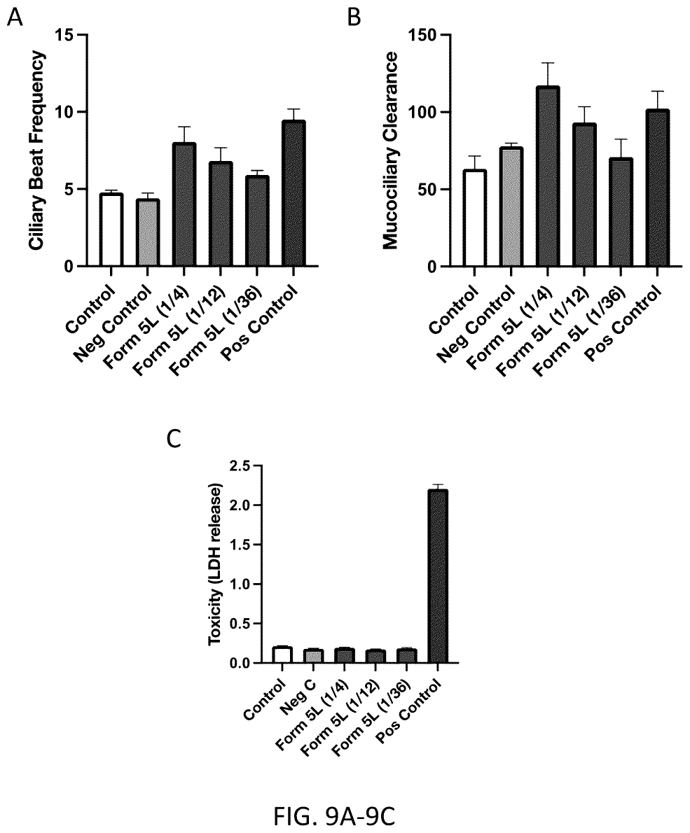

A summary of the results of each test epithelia assay is found in Table 8 (FIG. 9). Exposure with 10 μl of formulations 1 L, 2 L, 3 L and 5 L is well tolerated by the test epithelia (MucilAir™). No major signs of toxicity are observed. The comparator solution showed more cytotoxicity than the tested formulations. Each of the formulations appeared to show mucolytic effects at especially at 1:4 dilution compared to vehicle control, but results are similar with the comparator solution.

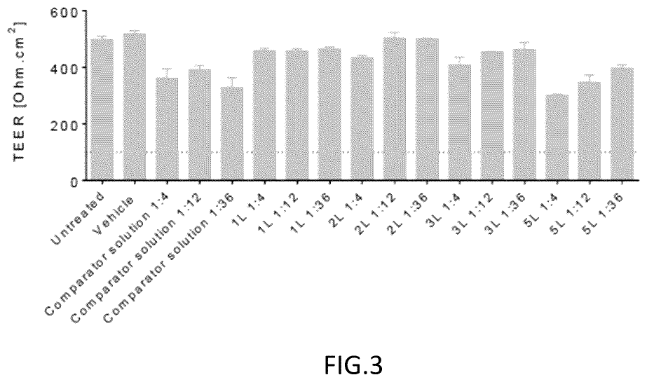

TEER—Tissue integrity is maintained for all tested compounds, at all tested concentrations. See FIG. 3. In FIG. 3, a TEER of 100 Ω·cm² is indicated with the dotted line. A TEER of below that value represents epithelia with low integrity.

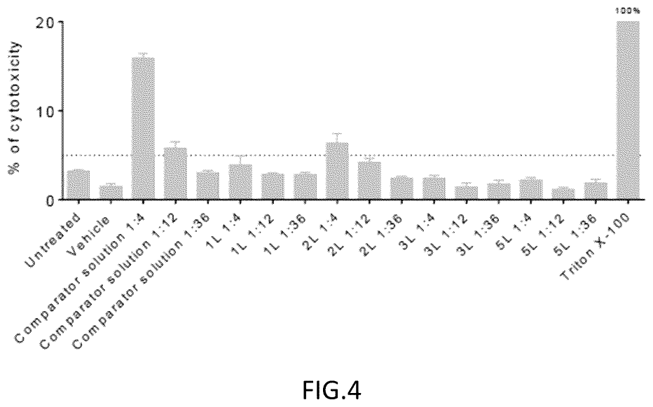

Cytotoxicity—No cytotoxicity is observed for the tested compounds, exceed for slight cytotoxicity of formulation 2 L at 1:4 dilution. However, the cytotoxicity of formulation 2 L at 1:4 dilution was less than the comparator solution at 1:4 dilution. There is a slight dose dependent cytotoxicity induced for the comparator solution. See FIG. 4. In FIG. 4, a % of toxicity of 5% is indicated with the dotted line. This dotted line represents an average LDH release due to cell turnover in the test epithelia.

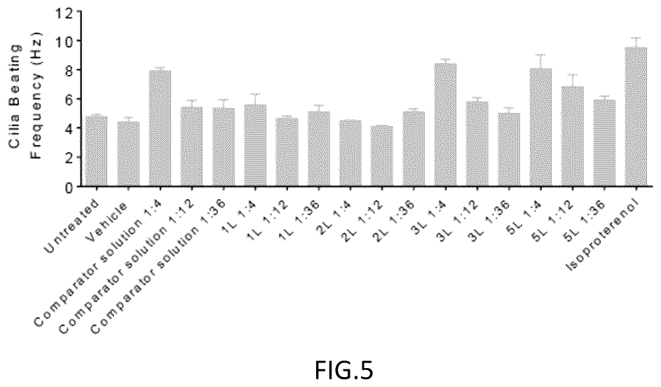

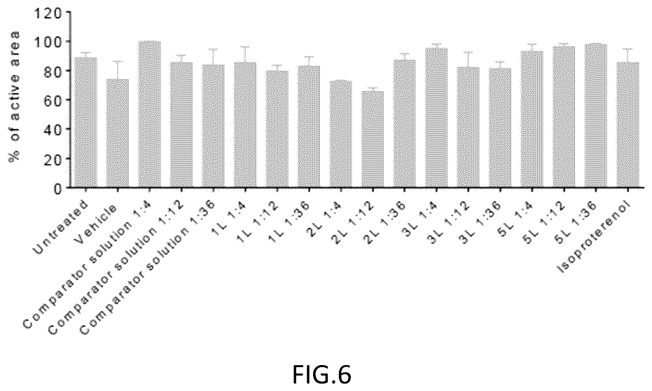

CBF—The positive control of cilia beating had the highest cilia beating frequency. Some tested compounds at 1:4 dilution, including the comparator solution, had higher beating frequencies. A slight increase of cilia beating frequency is observed for the formulations 3 L and 5 L, particularly at 1:4 and 1:12 dilutions compared to vehicle. A significant increase of cilia beating is also observed for the comparator solution at 1:4. In all treatments, there was no major modification observed for the % of active area of cilia beating measurements. See FIGS. 5 and 6.

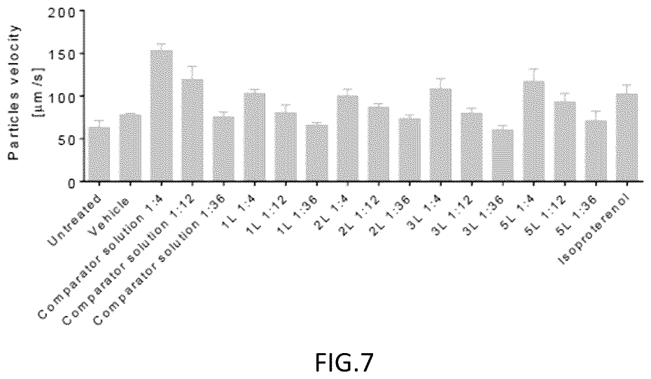

MCC—A dose dependent increase of mucociliary clearance is observed compared to vehicle control. The particles velocity is higher for all formulations at 1:4 dilution, especially for the comparator solution at 1:4 and 1:12 dilutions. See FIG. 7.

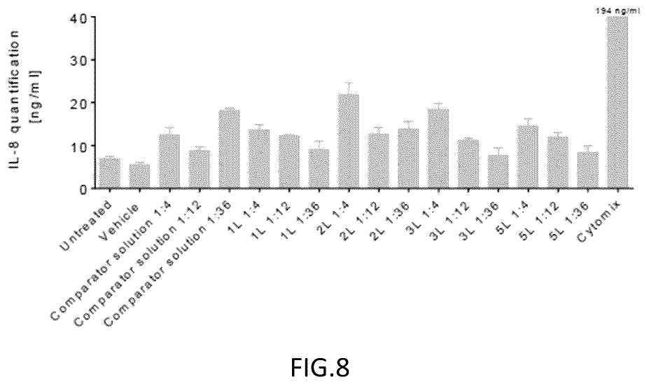

Basal secretion of IL-8—The positive control of inflammation, Cytomix, induced a strong secretion of basal IL-8 compared to the vehicle negative control (194 ng/ml vs<10 ng/ml). A dose dependent increase of IL-8 secretion is observed at 24 hours or all formulations but all values are below 20 ng/ml. See FIG. 8.

### Example 3: Formulations and their Characterization

Solutions for spray drying are prepared by weighing the ingredients as shown in Table 2 into a glass container and dissolving in 90 mL deionized water. The pH of the resulting solutions for formulations 1, 1 L, 1 HC L, 3 and 3 L are adjusted to pH 6.50±0.05 using 2M citric acid solution. The final volume of deionized water is added enough to make the total volume 100 mL. Dry powders of the solutions are produced by spray drying using a Pilotech YC-500 spray dryer with an inlet temperature of 130° C., fluid feed rate of 4 rpm, atomizing gas using air at 0.3 Mbar and air blower at 45 Hz.

Table 2: Ingredients of formulations. A number (e.g., 1, 2, 3 . . . ) indicates the base formulation, a number and the letter “L” indicates that the formulation is supplemented with leucine (e.g., 1 L, 4 L); a number and the letter “G” indicated that the formulation is supplemented with glycine; and a number and the letters “HC L” indicates that the formulation is supplemented with homocysteine and leucine. Thus, base formulation ingredient percentages can be deduced from a supplemented formulation. Citric acid control comprises about 15% sodium carbonate, about 75% cysteine, about 5% L-arginine and about 5% leucine.

TABLE 2

| | | Formulation | | | | | | | | |
|---|---|---|---|---|---|---|---|---|---|---|
| | | 1 | 1L | 1HC L | 2 | 3 | 4L | 4G | 5L | 6L |
| | Ingredients | % w/w | % w/w | % w/w | % w/w | % w/w | % w/w | % w/w | % w/w | % w/w |
| Formulation | Sodium carbonate | 13% | 12.80% | 12.8 | | 39% | 12.70% | 12.70% | 12.70% | 12.80% |
| Composition | citric acid | 15% | 15% | 14.7 | | 47.50% | 14.70% | 14.70% | 14.30% | 14.00% |
| | Cysteine | 67% | 63.80% | | 93.75% | | 63.70% | 63.70% | 63.30% | 63.80% |
| | homocysteine | | | 63.90% | | | | | | |
| | L-Arginine | 4% | 4.26% | 4.30% | 6.25% | 13% | 4.20% | 4.20% | 4.20% | 4.30% |
| | Forskolin | | | | | | 0.40% | 0.40% | 0.40% | |
| | leucine | | 4.26% | 4.309% | | | 4.20% | | 4.20% | 4.30% |
| | glycine | | | | | | | 4.20% | | |
| | Theobromine | | | | | | | | 0.80% | 0.80% |
| | RvD1 | | | | | | | | | |
| | RvE1 | | | | | | | | | |
| | Curcumin | | | | | | | | | |
| | Ellagic Acid | | | | | | | | | |

| | | Formulation | | | | | | | | |
|---|---|---|---|---|---|---|---|---|---|---|
| | | 9 | 10 | 11 | 12 | 13 | 14 | 15 | 16 | 18 |
| | Ingredients | % w/w | % w/w | % w/w | % w/w | % w/w | % w/w | % w/w | % w/w | |
| Formulation | Sodium carbonate | 10.40% | 10.40% | 21.70% | 21.90% | 22.60% | 17.71% | 21.90% | 21.80% | 31.50% |
| Composition | citric acid | 11.40% | 11.40% | 26.20% | 26.20% | 22.40% | 21.40% | 24.70% | 24.50% | 41.60% |
| | Cysteine | 51.80% | 51.80% | 36.20% | 36.50% | 37.70% | 29.52% | 36.50% | 36.20% | 2.80% |
| | homocysteine | | | | | | | | | |
| | L-Arginine | 3.50% | 3.50% | 7.20% | 7.30% | 7.50% | 5.90% | 7.30% | 7.30% | 10.50% |
| | Forskolin | 0.50% | 0.50% | 0.00% | 0.70% | 0.70% | 0.49% | 0.00% | 0.80% | 1.10% |
| | leucine | 3.50% | 3.50% | 7.20% | 7.30% | 7.50% | | 7.30% | 7.30% | 10.50% |
| | glycine | | | 0.00% | 0.00% | 0.00% | | 0.00% | 0.00% | |
| | Theobromine | 0.70% | 0.70% | 1.40% | 0.00% | 1.50% | 1.18% | 2.20% | 2.20% | 2.10% |
| | RvD1 | | 0.00% | | | | 0.00% | | | |
| | RvE1 | | 0.00% | | 0.00% | 0.00% | 0.00% | 0.00% | 0.00% | |
| | Curcumin | 13.40% | 13.40% | | 0.00% | 0.00% | 13.01% | 0.00% | 0.00% | |
| | Ellagic Acid | 5.00% | 5.00% | | | | 4.88% | | | |

A stock solution of forskolin was prepared by dissolving 10 mg of forskolin into 4 mL 200 proof ethanol. Solutions for spray drying were prepared by weighing the ingredients shown in Table 3 into a glass container and dissolving in 90 mL deionized water. The pH of the resulting solutions for formulations 4, 4 L, 4G, 5 and 5 L were adjusted to pH 6.50±0.05 using 2M citric acid solution. The final volume of deionized water is q.s. to 100 mL. Dry powders of the solutions are produced by spray drying using a Pilotech YC-500 spray dryer with an inlet temperature of 130° C., fluid feed rate of 4 rpm, atomizing gas using air at 0.3 Mbar and air blower at 45 Hz.

TABLE 3
Forskolin-containing formulations. Not being bound by any particular
theory, added forskolin is believed to increase mucociliary
clearance by speeding up ciliary beat frequency.

| Formulation | 4 (mg) | 4 L (mg) | 4 G (mg) | 5 (mg) | 5 L (mg) |
|---|---|---|---|---|---|
| Sodium Carbonate | 157.07 | 150.00 | 150.00 | 155.44 | 147.78 |
| Cysteine | 785.34 | 750.00 | 750.00 | 777.20 | 738.92 |
| L-Arginine | 52.36 | 50.00 | 50.56 | 51.80 | 49.26 |
| Leucine | — | 50.00 | — | — | 49.26 |
| Glycine | — | — | 50.80 | — | — |
| Forskolin Stock (2.5 | 2 mL | 2 mL | 2 mL | 1.979 mL | 1.9704 mL |
| mg/mL) | | | | | |
| Theobromine | — | — | — | 10.36 | 9.85 |
| Deionized Water | Q.S. 100 mL | Q.S. 100 mL | Q.S. 100 mL | Q.S. 100 mL | Q.S. 100 mL |

Aerosol Performance Characterization. Aerosol performance characterization of the spray dried powders was performed using a Next Generation Impactor (NGI) equipped with an induction port and without a pre-separator. Thirty milligrams of each formulation was weighed and loaded into size 3 inhalation grade hypromellose capsules. The capsules were pierced and actuated using a Plastiape RSO1 Model 7 medium resistance device. Impactor testing was performed at a flow rate of 80 L/min for a total of 4 L of air. The stages of the cascade impactor were coated using a 1.5% v/v mineral oil in isopropanol solution and allowed to dry to remove the isopropanol. After dose, each stage, the induction port, device, capsule and external filter were extracted with deionized water. The amount of formulation deposited at each position was calculated by measuring the uv absorbance at 224 nm using UV star (Grenier) 96 well plates and determining the resulting concentration from a calibration curve prepared with each formulation in deionized water.

TABLE 4
Aerosol Performance Characterization.

| | | Formulation | | | | | | | | | | |
|---|---|---|---|---|---|---|---|---|---|---|---|---|
| | | 1 | | 1L | | 1HC L | 4L | | 4G | 5L | | F13 |
| | | avg n = 3 | stdev | avg n = 3 | stdev | n = 1 | Avg = 3 | stdev | n = 1 | Avg n = 3 | stdev | N = 1 |
| Aerosol | Emitted | 18.69 | 1.19 | 24.49 | 2.00 | 31.91 | 23.17 | 2.62 | 24.73 | 23.71 | 1.04 | 15.51 |
| Performance | Dose (mg) | | | | | | | | | | | |
| (Cascade | Emitted | 61.45 | 3.08 | 79.22 | 5.45 | 96.45 | 74.92 | 4.53 | 76.71 | 80.70 | 3.88 | 62.04 |
| Impaction) | Fraction | | | | | | | | | | | |
| | (%) (based | | | | | | | | | | | |
| | on loading | | | | | | | | | | | |
| | dose) | | | | | | | | | | | |
| | RF <5 μm | 40.20 | 0.97 | 59.53 | 6.47 | 63.94 | 56.48 | 6.08 | 30.49 | 56.07 | 2.95 | 76.04 |
| | (%) based | | | | | | | | | | | |
| | on | | | | | | | | | | | |
| | recovered | | | | | | | | | | | |
| | dose | | | | | | | | | | | |
| | RF <3 μm | 31.94 | 0.74 | 48.56 | 6.13 | 47.37 | 42.17 | 4.30 | 16.36 | 44.37 | 2.67 | 63.45 |
| | (%) based | | | | | | | | | | | |
| | on | | | | | | | | | | | |
| | recovered | | | | | | | | | | | |
| | dose | | | | | | | | | | | |
| | MMAD (μm) | 3.00 | 0.11 | 2.23 | 0.18 | 2.80 | 2.75 | 0.24 | 7.98 | 2.54 | 0.07 | 2.17 |
| | GSD | 1.38 | 0.02 | 1.41 | 0.01 | 1.39 | 1.42 | 0.01 | 1.54 | 1.41 | 0.02 | 1.51 |

RF: respirable fraction; MMAD: mass-median aerodynamics diameter; GSD: geometric standard deviation. Emitted Fraction: the percentage that left the device; Respirable Fraction (RF) <5 micron: the percentage expected to deposit in the lungs; Respirable Fraction (RF) <3 micron: the percentage expected to deposit in the lungs.

Solutions for spray drying were prepared by weighing the ingredients as shown in Table 5 into a glass container and dissolving in 100 mL ethanol. Dry powders of the solutions are produced by spray drying using a Pilotech YC-500 spray dryer with an inlet temperature of 80° C., fluid feed rate of 4 rpm, atomizing gas using air at 0.3 Mbar and air blower at 45 Hz. Table 5 lists ethanol-based spray drying batches of the formulations that do not include the base formulation (1 L) but were processed in a separate step. Table 6 describes the compositions in which optional ingredients listed in Table 5 are blended with the base formulation to produce formulations with an array of agents that have various mechanisms of improving lung function.

TABLE 5
Formulations 7 and 8 and the optional ingredients therein.

| Formulation | 7 | 8 |
|---|---|---|
| Forskolin (mg) | 15 | 15 |
| Ellagic Acid (mg) | 150 | 150 |
| Curcumin (mg) | 400 | 400 |
| RvD1 (mg) | — | 0.1 |
| RvE1 (mg) | — | 0.1 |
| Ethanol | 100 mL | 100 mL |

Formulations 9 and 10 were prepared by dry blending spray dried formulations together. Each component was weighed as shown in Table 6 and placed into a 20 mL scintillation vial by geometric dilution and mixed by hand until homogeneous.

TABLE 6
Formulations 9 and 10. Formulation 6 L supplemented
with Formulation 7 (resulting in Formulation
9) or 8 (resulting in Formulation 10).

| Formulation | 9 | 10 |
|---|---|---|
| Formulation 6 L (mg) | 40.57 | 40.57 |
| Formulation 7 (mg) | 9.43 | — |
| Formulation 8 (mg) | — | 9.43 |

Particle Size Distribution. The particle size distribution of the prepared formulations was conducted by laser diffraction using a Sympatec Helos laser diffraction instrument equipped with an R3 lens and a RODOS powder dispersing unit set to a pressure of 4 bar. Powders were introduced to the laser diffractor beam using the RODOS rotating drum operated at 20 RPM. The particle size distribution was taken from measurements with an obscuration greater than 2%. Results are shown in Table 7.

TABLE 7
Volume distribution of particle within a size percentile.

| | Form | Form | Form | Form | Form | Form | Form | Form | Form | Form | Form |
|---|---|---|---|---|---|---|---|---|---|---|---|
| | 1 | 2 | 3 | 4 | 4L | 5 | 6L | 7 | 11 | 12 | 13 |
| Dv10(μm) | 0.99 | 1.01 | 1.00 | 1.52 | 1.22 | 1.42 | 0.97 | 0.67 | 0.81 | 0.77 | 0.93 |
| Dv50(μm) | 2.67 | 2.12 | 2.42 | 4.28 | 2.38 | 3.70 | 2.06 | 1.46 | 1.88 | 1.84 | 2.09 |
| Dv90(μm) | 6.86 | 3.80 | 15.04 | 22.50 | 4.16 | 10.37 | 3.64 | 3.08 | 3.51 | 3.61 | 3.84 |

Dv10 value (in μm) represents that 10% of particles in the formulation are smaller than this size; Dv50 value (in μm) represents that 50% of particles in the formulation are smaller than this size; Dv90 value (in μm) represents that 90% of particles in the formulation are smaller than this size. Between formulation 4 and 4 L, the addition of the leucine to a formulation decreases Dv90 to the target particle size range (less than 5 microns, with median size less than 3 microns) which can successfully deposit in the lung.

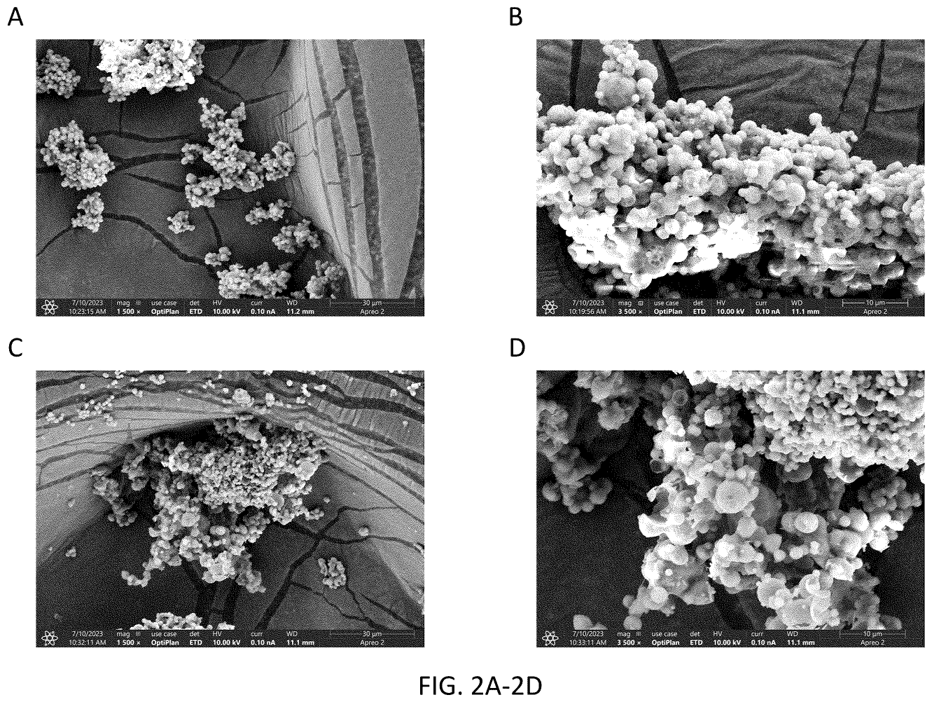

Scanning electron microscopy. Scanning electron microscopy was performed on Formulation 1 and Formulation 4. Samples were placed on carbon tape and sputter coated with gold for 2 minutes prior to analysis (Electron Microscopy Sciences). Images were taken at 1,500 and 3500 times magnification using an Apreo 2 (10.00 kV, 0.10 nA, Thermo Scientific). Results are shown in FIGS. 2A-2D.

The SEM results (FIGS. 2A-2D) confirm particle morphology and corroborate the laser diffraction results in Table 7. Laser diffraction assumes spherical particles in its calculations for particle size. SEM qualitatively confirmed this assumption.

Measuring Free Thiol of the Formulations. The percentage of free thiol in the formulations after spraying drying was conducted using a fluorometric free thiol assay kit (Abcam, ab112158). Preparations were weighed and diluted to a target free thiol content of 10 μM. The concentration of free thiol remaining in the formulation was determined based on a standard curve produced with glutathione.

Modeling mucociliary clearance. To model mucociliary clearance, sheep 40-50 kg were intubated and the velocity of mucociliary clearance in the trachea was measured by insufflation of radio-opaque Teflon discs were placed into the sheep trachea so that they become embedded in the mucus in the trachea. The movement of multiple discs over several minutes was determined using fluoroscopy and the tracheal mucus velocity (TMV) is calculated as the average that the discs travel per minute. After establishing a baseline TMV, a potent long-lasting inhibitor of CFTR (INH-172) was delivered by nebulization with TMV measurements performed every hour, and over a period of 2-3 hours the TMV declines by approximately 50% in a manner like CF where the inability to secrete Cl⁻ and NaHCO₃ results in dehydration of the airways and slowing of mucociliary clearance.

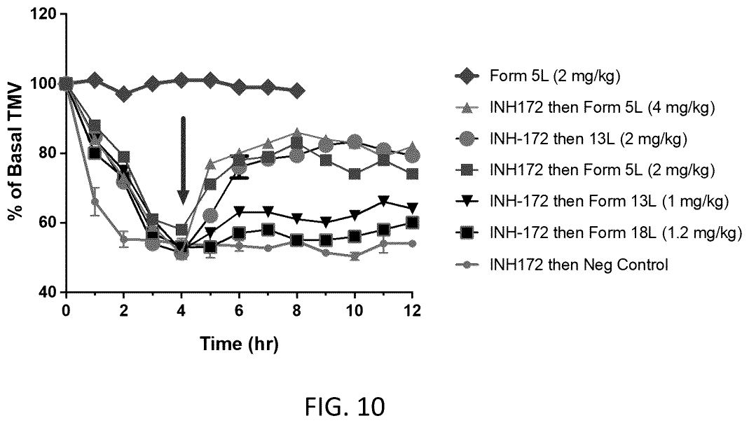

Once the stable inhibition of TMV has been established, typically at hour 4, the test compound was delivered by nebulization and TMV measured for an additional 8 hours. Inhibitors of ENaC like amiloride (as described in Scott, David W., et al., American Journal of Respiratory and Critical Care Medicine 196.6 (2017): 734-744, incorporated herein in its entirety) and P1037/VX371 show rescue of TMV that is of limited duration with an effect that only lasts for 2-3 hours, due to systemic clearance. By contrast, the experimental formulations disclosed herein demonstrated rescue of TMV that persisted until the end of the evaluation period at hour 12 because the effect is on the mucus and not due to short-term stimulation of ion channels. See FIG. 10.

### EQUIVALENTS

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control. Other embodiments are set forth within the following claims.

1. A composition comprising:
(a) a thiol-containing reducing agent;
(b) citric acid or a physiologically acceptable salt thereof;
(c) carbonic acid or a physiologically acceptable salt thereof; and
(d) a stabilizer.

2. The composition of claim 1, wherein the thiol-containing reducing agent comprises cysteine, N-Acetyl cysteine, homocysteine, dihydrolipoic acid, glutathione, coenzyme A, ergothioneine, mycothiol, bacillithiol, or combinations thereof.

3. The composition of claim 1, wherein the stabilizer comprises leucine, lecithin, magnesium stereate, trileucine, isoleucine, glycine, lactose, mannitol, trehalose, erythritol, sorbitol, or combinations thereof.

4. The composition of claim 1, wherein the composition comprises (a) 40%-80% (w/w) of a thiol-containing reducing agent; (b) 8%-20% (w/w) of citric acid or a physiologically acceptable salt thereof; (c) 8%-20% (w/w) of carbonic acid or a physiologically acceptable salt thereof; and (d) 3%-6% (w/w) of a stabilizer.

5. The composition of claim 1, wherein the pH of the composition ranges from about 5.5 to about 10, about 5.5 to about 8, about 6 to about 10, or about 6 to about 8.

6. The composition of claim 1, wherein the composition comprises an aqueous solution or a dry powder.

7. The composition of claim 1, wherein the composition is suitable for administration by inhalation.

8. The composition of claim 1, further comprising at least one ingredient selected from Adhatoda vasica Nees extract, Albizzia lebbeck (Sareesha rakat) extract, Alstonia scholaris extract, Artemisia caerulescens extract, Belamcanda chinensis extract, Benincasa hispida extract, Cissampelos sympodialis extract, Clerodendron serratum extract, Coleus forskohlii extract, Elaeocarpus spharicus extract, Galphimia glauca extract, Gardenia latifolia extract, Gingko biloba extract, Mikania glomerata extract, Lepidium sativum extract, Ocimum sanctum extract, Passiflora incarnata extract, Pavetta crassipes extract, Picrorrhiza kurroa extract, Sarcostemma brevistigma extract, Tephrosia purpurea extract, Tylophora indica extract, Vitex negundo extract, Rosmarinus officinalis extract, Ephedra sinica extract, and Gleditsia sinensis Lam extract.

9. The composition of claim 1, further comprising at least one ingredient selected from bicarbonate, Ascorbic acid, EDTA, N-acetylcysteine, Caffeine citrate, L-Arginine, Forskolin, Theobromine, resolving D1 (RvD1), resolving E1 (RvE1), lipoxin A1 (LXA4), lipoxin B4 (LXB4), protectin D1 (PD1), maresin 1 (MaR1), maresin 2 (MaR2), 17-HDHA, 14-HDHA, ellagic acid, curcumin, 18-HEPE, or a combination thereof.

10. The composition of claim 1, wherein the composition comprises a lyophilized dry powder.

11.-14. (canceled)

15. A method of alleviating one or more negative effects of a pulmonary or airway disorder comprising administering to a subject in need thereof a composition arising from a combination of a thiol-containing reducing agent, citric acid or a physiologically acceptable salt thereof, carbonic acid or a physiologically acceptable salt thereof, and a stabilizer.

16. The method of claim 15, wherein the pulmonary or airway disorder is selected from chronic inflammatory lung disease, pulmonary fibrosis, pulmonary vasculitis, pulmonary sarcoidosis, inflammation and/or infection associated with lung transplantation, acute or lung rejection and/or dysfunction, pulmonary artery hypertension, bronchitis, sinusitis, asthma, cystic fibrosis, bronchiectasis, bacterial infection, fungal infection, parasite infection, viral infection, chronic obstructive pulmonary disease (COPD), bronchiolitis obliterans syndrome (BOS), primary ciliary dyskinesia (PCD), alveolar proteinosis, idiopathic pulmonary fibrosis, eosinophilic pneumonia, eosinophilic bronchitis, acute respiratory distress syndrome (ARDS), inflammation and/or infection associated with mechanical ventilation, ventilator-associated pneumonia, asbestos-related airway disorder or disease, dust-related airway disorder or disease, silicosis, and radiation or chemical agent-related airway disease or disorder, and any combination thereof.

17. A method of enhancing clearance of pollutants, microparticulate matter, or other irritants from the lungs of a subject comprising administering to a subject in need thereof a composition arising from a combination of a thiol-containing reducing agent, citric acid or a physiologically acceptable salt thereof, carbonic acid or a physiologically acceptable salt thereof, and a stabilizer.

18. A method of cleansing at least a portion of the lungs of a subject comprising administering to at least a portion of the lungs of a subject in need thereof a composition arising from a combination of a thiol-containing reducing agent, citric acid or a physiologically acceptable salt thereof, carbonic acid or a physiologically acceptable salt thereof, and a stabilizer.

19. The method of claim 17, wherein the composition is a dry powder administered through inhalation.

20. The method of claim 17, wherein the composition is administered by a passive dry powder inhaler (DPI).

21. The method of claim 17, wherein the composition is administered at least once a day, twice a day, three times a day, or as needed.

22. The method of claim 17, wherein the combination excludes divalent cations.

23. The method of claim 17, wherein the composition further comprises a pharmacologically acceptable carrier.

24. The method of claim 17, in which said at least a portion of the lungs is cleansed of at least one irritant selected from a cigarette smoke, bacteria, a mold, dust mites, acarids, a pollen, a material from an insect, a material from an animal and animal dander, fungi, an air pollutant, an irritant gas, an aerosol, a vapor, a fume, a noxious chemical, or combinations thereof.

25. (canceled)

26. The method of claim 17, wherein the composition further comprises at least one ingredient selected from Adhatoda vasica Nees extract, Albizzia lebbeck (Sareesha rakat) extract, Alstonia scholaris extract, Artemisia caerulescens extract, Belamcanda chinensis extract, Benincasa hispida extract, Cissampelos sympodialis extract, Clerodendron serratum extract, Coleus forskohlii extract, Elaeocarpus spharicus extract, Galphimia glauca extract, Gardenia latifolia extract, Gingko biloba extract, Mikania glomerata extract, Lepidium sativum extract, Ocimum sanctum extract, Passiflora incarnata extract, Pavetta crassipes extract, Picrorrhiza kurroa extract, Sarcostemma brevistigma extract, Tephrosia purpurea extract, Tylophora indica extract, Vitex negundo extract, Rosmarinus officinalis extract, Ephedra sinica extract, and Gleditsia sinensis Lam extract.

27. The method of claim 17, wherein the composition further comprises at least one ingredient selected from bicarbonate, Ascorbic acid, EDTA, N-acetylcysteine, Caffeine citrate, L-Arginine, Forskolin, Theobromine, resolving D1 (RvD1), resolving E1 (RvE1), lipoxin A1 (LXA4), lipoxin B4 (LXB4), protectin D1 (PD1), maresin 1 (MaR1), maresin 2 (MaR2), 17-HDHA, 14-HDHA, ellagic acid, curcumin, 18-HEPE, or a combination thereof.

28. The method of claim 17, wherein the thiol-containing reducing agent comprises cysteine, N-Acetyl cysteine, homocysteine, dihydrolipoic acid, glutathione, coenzyme A, ergothioneine, mycothiol, bacillithiol, or combinations thereof.

29. The method of claim 17, wherein the stabilizer comprises leucine, lecithin, magnesium stereate, trileucine, isoleucine, glycine, lactose, mannitol, trehalose, erythritol, sorbitol, or combinations thereof.

30. A composition comprising a mixture arising from a combination of: (a) a thiol-containing reducing agent; (b) citric acid; (c) carbonic acid; (d) a stabilizer, or one or more physiologically acceptable salt of any of the foregoing.
